SEQUENCE LISTING (18 sequences)

SEQ ID NO: 1 - DNA, 352 bp, Homo sapiens
cctggacctagaaacaaaaaatgaaagtggagaaaaccaaacaagaatctttctttcttaaagtaaacatcggactgacatctcctttcaaacattcattgaggatctactctgtgagaagaagcaataccatgttatctcattctagttccagttctagttgacttgattgtcaaacgactgctaagaaggtgaaatagaggagatgacatgaatgagggactacattggaatgaagaagaacctggaggaggtaaaaaaactgaaggctccggatgtcggacgatggaaaataaaaacaacaagcaagtgaaactccctgacttatcaaccttactcaagaattccttct

SEQ ID NO: 2 - RNA, 19 nt, Artificial Sequence
  siRNA
gacuuaucaaccuuacuca

SEQ ID NO: 3 - RNA, 19 nt, Artificial Sequence
  siRNA
ugaguaagguugauaaguc

SEQ ID NO: 4 - RNA, 19 nt, Artificial Sequence
  siRNA
cauguuaucucauucuagu

SEQ ID NO: 5 - RNA, 19 nt, Artificial Sequence
  siRNA
acuagaaugagauaacaug

SEQ ID NO: 6 - RNA, 19 nt, Artificial Sequence
  siRNA
cugacaucuccuuucaaac

SEQ ID NO: 7 - RNA, 19 nt, Artificial Sequence
  siRNA
guuugaaaggagaugucag

SEQ ID NO: 8 - DNA, 1521 bp, Homo sapiens
cttccacatcgtggaagctttgttctttcgctctttgcaataaatcttgctactgctcactctttgggtccacgctgcttttatgagctgtaacactcaccgcgaagatctgcagcttcactcccgagccagcgagaccacgaacccaccagaaggaagaaactccgaacacatctgaacatcagaagggcagactccagacacgccaccttaagagctgtaacactcaccgcgagggtccacggcttcattcttgaagtcagtgagaccaagaacccaccaattccggacacaatttcttggctctcagtggcttccatggaagttctgcaatcaaccagcaggagaaccgcttaaacccaggaggcggaggttgcagtgagccaagtatgcatcactgcactccagcctggaagacagagtgagaccctgtctcaacaaaataaattaaaataaaaaataatatatttttctaactatcatcccttttccaaatcaggaattccccttaagttttcctcaatttccatggcaatatctttgcatagattcattaagaatttgtcctttttaaataaaaaatataaagggaactattcattaggcaacaaatgccttgtctgaaatatcacatttgagaatgctgctcatttaatcagaaaggtacgctactttaaagaactgaggtccactttctggagccaaaaactcataaatccctctcagaaaaacctgatttgctttgtagggtctcaggtttagagatgctgaaaaagatattttcgttgcagacaaaggacctcagagtatttggagaactttgagaagagaggaattctcccaaatgtataggtgtcacaggtaaaatacagtcgagagattttcttggactttaattccttaaatcaggatagcaaataataggggcttttacaaattcaatctgtttccttacaaaaattttcagcaaagtaatttcagcaaattaattttcagcaaagtaaatgtaagaagacttatgtgaaaaattaacattctccatgtatctatgaagccaaacctaataaaaccagctttaatttgtgctcaagaatattatttcactgagttttcttaaatcacaaaggggagactgttatgaaaactgatataaaataaaaaaaacaaggaagaaaagtctactagatgtctctaatggaagactgcatttttagaacatatccttataggcgattctagcctttctctgctatttggctctcacactctttaccgtgcagataattcacagcaatgcaaaagaatcctcatctatagccatgaaaataagttatttgttatttctggtaaaggttcaattgacctccccttccaggatgaagaaagtttcatgtctttctgcatcatttcaactattccttactacatataaatctgcacttgttaacttctattttgaattgattgtggcatctgcctgcttccccattaaaactgaataaaatctttaacacataaaaa

SEQ ID NO: 9 - DNA, 20 bp, Artificial Sequence
  ASO
  misc_feature (1)..(5): 2-O-Me RNA base
  misc_feature (1)..(20): Phosphorothioate bonds
  misc_feature (16)..(20): 2-O-Me RNA base
guguctggagtctgcccuuc

SEQ ID NO: 10 - DNA, 20 bp, Artificial Sequence
  AOS
  misc_feature (1)..(5): 2-O-Me RNA base
  misc_feature (1)..(20): Phosphorothioate bonds
  misc_feature (16)..(20): 2-O-Me RNA base
gaagcaggcagatgccacaa

SEQ ID NO: 11 - DNA, 20 bp, Artificial Sequence
  AOS
  misc_feature (1)..(5): 2′-O-Me RNA base
  misc_feature (1)..(20): Phosphorothioate bonds
  misc_feature (16)..(20): 2-O-Me RNA base
aggucctttgtctgcaacga

SEQ ID NO: 12 - DNA, 10 bp, Artificial Sequence
  Oligonucleotide
gcagactcca

SEQ ID NO: 13 - DNA, 10 bp, Artificial Sequence
  Oligonucleotide
gcatctgcct

SEQ ID NO: 14 - DNA, 10 bp, Artificial Sequence
  Oligonucleotide
gcagacaaag

SEQ ID NO: 15 - RNA, 19 nt, Artificial Sequence
  Oligonucleotide
accaaauguacagcugauu

SEQ ID NO: 16 - RNA, 19 nt, Artificial Sequence
  Oligonucleotide
accaaauguacaacacacu

SEQ ID NO: 17 - RNA, 19 nt, Artificial Sequence
  Oligonucleotide
accaaauguacaaaagacu

SEQ ID NO: 18 - RNA, 19 nt, Artificial Sequence
  Oligonucleotide
accaaauguacaaaaggau

(19) United States
(12) Patent Application Publication
(10) Pub. No.: US 2022/0135979 A1
(43) Pub. Date: May 5, 2022

(54) DIAGNOSIS AND TREATMENT OF MEDULLOBLASTOMA

(71) Applicant: TEL HASHOMER MEDICAL RESEARCH INFRASTRUCTURE AND SERVICES LTD, Ramat Gan (IL)

(72) Inventors: Ruty SHAI, Kfar Saba (IL); Shany FREEDMAN PICCIOTTO, Tel Aviv (IL); Amos TOREN, Tel Aviv (IL)

(21) Appl. No.: 17/430,072

(22) PCT Filed: Feb. 27, 2020
(86) PCT No.: PCT/IL2020/050229

§ 371 Date: Aug. 11, 2021

Related U.S. Application Data
(60) Provisional application No. 62/810,981, filed on Feb. 27, 2019.
(60) Provisional application No. 62/810,984, filed on Feb. 27, 2019.

(51) Int. Cl. C12N 15/113; C12Q 1/6806; C12Q 1/6851; C12Q 1/6886; A61K 31/713; A61P 35/00

(52) U.S. Cl. CPC C12N 15/1135; C12Q 1/6806; C12Q 1/6851; C12Q 1/6886; C12N 2310/14; A61P 35/00; C12N 2320/30; C12Q 2600/112; C12Q 2600/158; A61K 31/713

(57) ABSTRACT

Methods for classifying, identifying and monitoring of a medulloblastoma tumor in a subject are provided, based on lincRNA expression in the tumor and/or in a biological sample of the subject. Compositions and methods for treating medulloblastoma using inhibitors of the specific lincRNAs disclosed herein. Additionally, novel inhibitory nucleic acids targeting the specific lincRNAs are provided.

### FIELD OF THE INVENTION

The present invention relates to classification, identification and monitoring of a medulloblastoma tumor in a subject based on lincRNA expression in the tumor and/or in a biological sample of the subject. The present invention further relates to treatment of medulloblastoma using inhibitors targeting specific lincRNAs identified to be over-expressed in the tumor, and to novel inhibitory nucleic acids targeting the specific lincRNAs.

### BACKGROUND OF THE INVENTION

Medulloblastoma (MB) is the most common malignant pediatric brain tumor. The histological entity known as medulloblastoma is comprised of multiple clinically and molecularly distinct subgroups with the current consensus of four defined core groups: wingless-activated (WNT), sonic hedgehog-activated (SHH), Group 3, and Group 4, each characterized by specific mutations, copy number alterations, transcriptomic/methylomic profiles, and clinical outcomes. Group assignment is prognostic, where WNT tumors have the most favorable prognosis. Group 3 tumors, generally restricted to the pediatric age, are the most-serious owing to a very poor prognosis, with many patients dying despite aggressive therapy. This group has the highest rates of metastases, frequently already at the time of diagnosis. SHH and Group 4 medulloblastomas have an intermediate prognosis, where Group 4 accounts for 25% of adult medulloblastomas, often with a worse prognosis than in childhood cases (Northcott et al., Nat. Rev. Neurol. (8):340-351, 2012). Group 3 and Group 4 are more related to each other than to WNT and SHH and appear as non-WNT/non-SHH in the revised 2016 WHO classification (Louis et al., Acta Neuropathol. 131:803-820,2016).

The standard of care for MB consists of surgery, chemotherapy and age-dependent radiation therapy. At present, treatment intensity for medulloblastoma is based on clinical biomarkers, such as, age, extent of resection, the presence of metastases, and pathological diagnosis of specific histological variants of the disease. Medulloblastoma tumors are very fast-growing, they often spread to other areas of the central nervous system (CNS) through cerebrospinal fluid (CSF). Approximately 30-35% of the patients present evidence of a metastatic disease. These medulloblastoma patients remain incurable after treatment, and are associated with significant morbidity and mortality enhanced by the neurotoxicity developed in the brain, which is caused by chemotherapy.

Early detection of relapse and precise diagnosis has direct prognostic relevance if treated appropriately with irradiation-sparing regimen. Despite recent advances in treatment, 30% to 40% of children which are high risk patients, experience tumor recurrence, and most of them die from disease especially if previously irradiated during therapy.

Long Intergenic Noncoding (linc or lnc) RNAs, also known as long non-coding RNA, is a class of non-coding RNAs (ncRNAs) that range in size from several hundred to tens of thousands base pairs. LincRNAs have an impact on various human diseases, including cancer.

There is an unmet need for accurate diagnostic assays and more efficient treatment of medulloblastoma.

### SUMMARY OF THE INVENTION

The present disclosure provides nucleic acids and compositions which may be used in methods for classifying, identifying, monitoring and treatment of medulloblastoma tumors.

Long Intergenic Noncoding (linc or lnc) RNAs, also known as long non-coding RNA, is a class of non-coding RNAs (ncRNAs) that range in size from several hundred to tens of thousands base pairs. LincRNAs have an impact on various human diseases, including cancer. The present disclosure provides a lincRNA set forth in SEQ ID NO: 1, also identified as “MB3 lincRNA”, as a biomarker of Group 3 medulloblastoma and metastatic medulloblastoma, and a lincRNA set forth in SEQ ID NO: 8, also identified as “MB4 lincRNA”, as a biomarker of non-WNT/non-SHH medulloblastoma.

As further disclosed herein, MB4 lincRNA over-expression in a biological fluid sample of a subject, such as a plasma sample, is indicative of the presence of a tumor and can be used for monitoring tumor recurrence and/or progression. It was surprisingly found that in plasma samples of medulloblastoma patients in which the tumor recurred following resection, an increase in the level of MB4 lincRNA was detected several months before the tumor was detected by an MRI scan, thus providing a highly useful marker for monitoring recurrence and for prognosis of MB4-related medulloblastomas.

The present invention further provides compositions and methods for treating medulloblastoma using inhibitors of lincRNAs, as disclosed herein. Additionally, novel inhibitory nucleic acids targeting lincRNAs are provided. Advantageously, the lincRNAs disclosed herein serve as specific targets for suppressing and/or inhibiting tumor progression and metastasis in medulloblastomas, for example medulloblastomas of the poorest prognosis.

Surprisingly, as exemplified hereinbelow, silencing of MB3 lincRNA (SEQ ID NO: 1) impairs MB cellular migration and invasion while over-expression of this lincRNA enhances MB cellular migration and invasion. Moreover, silencing of MB3 lincRNA leads to a decrease in THSD7A expression in MB cells and to a decrease in the phosphorylation of Focal Adhesion Kinase (FAK), thereby inhibiting tumor cell migration. Overexpression of MB3 lincRNA enhanced the expression of THSD7A and enhanced FAK phosphorylation which enhance invasion, migration and angiogenesis. MB3 lincRNA is provided herein as a strong diagnostic biomarker and a valuable therapeutic target for inhibition of invasive (metastatic) medulloblastoma.

According to one aspect, the present invention provides a method for classifying a medulloblastoma tumor of a subject, the method comprising measuring a level of at least one lincRNA selected from the group consisting of MB3 lincRNA (SEQ ID NO: 1) and MB4 lincRNA (SEQ ID NO: 8) in a sample of the medulloblastoma tumor,

wherein a level of MB3 lincRNA above a predefined threshold is indicative of at least one of Group 3 medulloblastoma and metastatic medulloblastoma; and

wherein a level of MB4 above a predefined threshold is indicative of non-WNT/non-SHH medulloblastoma.

In some embodiments, the level of the at least one lincRNA is measured by extracting RNA from said tumor sample, reverse-transcribing the RNA to cDNA, and subjecting the cDNA to quantitative amplification for quantifying the at least one lincRNA.

In some embodiments, a method for treating a subject with a medulloblastoma tumor is provided, the method comprising:

(i) classifying the medulloblastoma tumor of the subject as Group 3 and/or metastatic medulloblastoma according to the method of the present invention, by detecting a level of MB3 lincRNA above a predefined threshold in a sample of the tumor; and

ii) administering an MB3 lincRNA inhibitor to the subject.

In some embodiments, a method for treating a subject with a medulloblastoma tumor is provided, the method comprising:

(i) classifying the medulloblastoma tumor of the subject as non-WNT/non-SHH medulloblastoma according to the method of the present invention, by detecting a level of MB4 lincRNA above a predefined threshold in a sample of the tumor; and

(ii) administering an MB4 lincRNA inhibitor to the subject.

In some embodiments, the inhibitor is an inhibitory nucleic acid. In some embodiments, the inhibitory nucleic acid is selected from the group consisting of small-inhibitory RNA (siRNA), short hairpin RNA (shRNA), microRNA (miRNA) and antisense oligonucleotide (ASO), or a vector encoding the same. Each possibility represents a separate embodiment of the present invention.

According to another aspect, the present invention provides a method for monitoring non-WNT/non-SHH medulloblastoma in a subject previously diagnosed with non-WNT/non-SHH medulloblastoma, the method comprising:

(i) obtaining a plurality of biological samples comprising circulating RNA taken from the subject over a period of time; and

(ii) measuring a level of MB4 lincRNA (SEQ ID NO: 8) in the plurality of biological samples, wherein an increase in the level of MB4 lincRNA over the period of time is indicative of progression or recurrence of non-WNT/non-SHH medulloblastoma in the subject.

According to an additional aspect, the present invention provides a method for identifying non-WNT/non-SHH medulloblastoma in a subject, the method comprising measuring a level of MB4 lincRNA (SEQ ID NO: 8) in a biological sample comprising RNA taken from the subject, wherein a level above a predefined threshold is indicative of non-WNT/non-SHH medulloblastoma in the subject.

In some embodiments, the biological sample is selected from the group consisting of blood, plasma and serum.

In some embodiments, measuring a level of MB4 lincRNA is carried out by an amplification or hybridization method. Each possibility represents a separate embodiment of the present invention. In some particular embodiments, the amplification method is droplet digital PCR (ddPCR).

According to an additional aspect, the present invention provides a method of treating medulloblastoma in a subject in need thereof, the method comprising:

(i) measuring a level of MB3 lincRNA in a medulloblastoma tumor sample obtained from the subject:

(ii) detecting a level of MB3 lincRNA above a control level; and

(ii) administering to the subject an MB3 lincRNA inhibitor.

In some embodiments, the MB3 lincRNA inhibitor is an inhibitory nucleic acid targeting MB3 lincRNA. In some embodiments, the inhibitory nucleic acid targeting MB3 lincRNA is a small-inhibitory RNA (siRNA), short hairpin RNA (shRNA), microRNA (miRNA), or antisense oligonucleotide (ASO), or a vector encoding the same. Each possibility represents a separate embodiment of the present invention.

In some particular embodiments, the inhibitory nucleic acid targeting MB3 lincRNA is an siRNA or a vector encoding the same selected from the group consisting of:

(a) siRNA with a sense sequence as set forth in SEQ ID NO: 3 and an antisense sequence as set forth in SEQ ID NO: 3; (b) siRNA with a sense sequence as set forth in SEQ ID NO: 4 and an antisense sequence as set forth in SEQ ID NO: 5; (c) siRNA with a sense sequence as set forth in SEQ ID NO: 6 and an antisense sequence as set forth in SEQ ID NO: 7; and combinations thereof. Each possibility represents a separate embodiment of the present invention.

According to a further aspect, the present invention provides a synthetic double-stranded RNA targeting MB3 lincRNA or a vector encoding the same, selected from the group consisting of:

(A) a dsRNA comprising a sense strand comprising the sequence set forth in SEQ ID NO: 2 and an antisense strand comprising the sequence set forth in SEQ ID NO: 3;

(B) a dsRNA comprising a sense strand comprising the sequence set forth in SEQ ID NO: 4 and an antisense strand comprising the sequence set forth in SEQ ID NO: 5; and

(C) a dsRNA comprising a sense strand comprising the sequence set forth in SEQ ID NO: 6 and an antisense strand comprising the sequence set forth in SEQ ID NO: 7, wherein each strand is between 19 to 25 nucleotides in length.

In some embodiments, a pharmaceutical composition is provided, comprising the synthetic dsRNA of the present invention and a pharmaceutically acceptable carrier.

In some embodiments, a method for treating medulloblastoma in a subject in need thereof is provided, the method comprising administering to the subject the synthetic dsRNA of the present invention, or a composition comprising the same.

According to a further aspect, the present invention provides a method for treating medulloblastoma, the method comprising administering to a subject with medulloblastoma an inhibitory nucleic acid targeting MB3 lincRNA.

In some embodiments, the medulloblastoma is characterized by over-expression of MB3 lincRNA.

In some embodiments, the inhibitory nucleic acid targeting MB3 lincRNA is a small-inhibitory RNA (siRNA), short hairpin RNA (shRNA), microRNA (miRNA), antisense oligonucleotide (ASO) or a vector encoding the same. Each possibility represents a separate embodiment of the present invention.

In some particular embodiments, the inhibitory nucleic acid targeting MB3 lincRNA is an siRNA or a vector encoding the same selected from the group consisting of: (a) siRNA with a sense sequence as set forth in SEQ ID NO: 3 and an antisense sequence as set forth in SEQ ID NO: 3; (b) siRNA with a sense sequence as set forth in SEQ ID NO: 4 and an antisense sequence as set forth in SEQ ID NO: 5; c) siRNA with a sense sequence as set forth in SEQ ID NO: 6 and an antisense sequence as set forth in SEQ ID NO: 7; and combinations thereof. Each possibility represents a separate embodiment of the present

These and further aspects and features of the present invention will become apparent from the detailed description, examples and claims which follow.

### BRIEF DESCRIPTION OF THE FIGURES

FIG. 1. Differential expression of MB3 lincRNA (SEQ ID NO: 1) in the four medulloblastoma core groups (n=61).

FIG. 2A. Expression of MB3 lincRNA (SEQ ID NO: 1) in medulloblastoma DAOY cells treated with siRNAs set forth in SEQ ID NOs: 2-7.

FIG. 2B. Expression of MB3 lincRNA (SEQ ID NO: 1) in medulloblastoma UW-228 cells treated with siRNAs set forth in SEQ ID NOs: 2-7.

FIG. 2C. Expression of MB3 lincRNA (SEQ ID NO: 1) in medulloblastoma DAOY cells treated with each of siRNA1 (SEQ ID NOs: 2-3), siRNA2 (SEQ ID NO: 4-5) and siRNA3 (SEQ ID NO: 6-7), or with different combinations of these siRNAs.

FIG. 3A. Representative images of UW-228 and DAOY cells that invaded matrigel coated transwells, after incubation with the siRNAs set forth in SEQ ID NOs: 2-7 or with a control siRNA.

FIG. 3B. Graphical representation of the results of an invasion assay performed with DAOY cells cultured with the siRNAs set forth in SEQ ID NOs: 2-7 or with a control siRNA.

FIG. 3C. Graphical representation of the results of an invasion assay performed with UW-228 cells cultured with the siRNAs set forth in SEQ ID NOs: 2-7 or with a control siRNA.

FIG. 4A. Representative images of confluent UW-228 and DAOY cell cultures that were scratched and incubated for 24 hours with the siRNAs set forth in SEQ ID NOs: 2-7 or with a control siRNA.

FIG. 4B. Graphical representation of the results of a migration assay performed with confluent DAOY cells cultures that were scratched and incubated for 24 hours with the siRNAs set forth in SEQ ID NOs: 2-7 or with a control siRNA (n=3).

FIG. 4C. Graphical representation of the results of a migration assay performed with confluent UW-228 cells cultures that were scratched and incubated for 24 hours with the siRNAs set forth in SEQ ID NOs: 2-7 or with control siRNA (n=3).

FIG. 5A. Representative images of UW-228 and DAOY cells over-expressing the MB3 lincRNA set forth in SEQ ID NO: 1, or the corresponding control cells, that invaded matrigel coated transwells.

FIG. 5B. Graphical representation of the results of an invasion assay performed with DAOY cells over-expressing the MB3 lincRNA set forth in SEQ ID NO: 1 and control DAOY cells (n=3).

FIG. 5C. Graphical representation of the results of an invasion assay performed with UW-228 cells over-expressing the MB3 lincRNA set forth in SEQ ID NO: 1 and control UW-228 cells (n=3).

FIG. 6A. Representative images of confluent scratched cultures (migration assay) of UW-228 and DAOY cells over-expressing the MB3 lincRNA set forth in SEQ ID NO: 1, or the corresponding control cells.

FIG. 6B. Graphical representation of the results of a migration assay performed with DAOY cells over-expressing the MB3 lincRNA set forth in SEQ ID NO: 1, or the corresponding control cells (n=3).

FIG. 6C. Graphical representation of the results of a migration assay performed with UW-228 cells over-expressing the MB3 lincRNA set forth in SEQ ID NO: 1, or the corresponding control cells (n=3).

FIG. 7A. THSD7A expression following silencing of MB3 lincRNA in DAOY cells compared to si negative (p=0.03).

FIG. 7B. THSD7A expression following silencing of MB3 lincRNA in UW-228 cells compared to si negative (p=0.03).

FIG. 8A. THSD7A expression following over-expression of MB3 lincRNA in DAOY cells compared to empty plasmid (p=0.033).

FIG. 8B. THSD7A expression following over-expression of MB3 lincRNA in UW-228 cells, compared to empty plasmid (p=0.06).

FIG. 8C. THSD7A protein level in the medium of DAOY and UW-228 cells over-expressing MB3 lincRNA compared to the medium alone (MEM) and to the medium of cells transfected with an empty plasmid (Empty).

FIG. 9A. Images of DAOY cells over-expressing MB3 lincRNA (right panel) or transfected with an empty plasmid (left panel), stained for THSD7A.

FIG. 9B. Graphical representation corresponding to FIG. 9A, of the relative percentage of THSD7A stained DAOY cells.

FIG. 10A Representative images of cells stained for THSD7A following siRNA silencing of MB3 lincRNA compared to control (si-negative).

FIG. 10B. Relative percentage of THSD7A stained DAOY cells in which MB3 lincRNA was silenced using siRNA compared to control (si-negative).

FIG. 10C. Relative percentage of THSD7A stained UW-228 cells in which MB3 lincRNA was silenced using siRNA compared to control (si-negative).

FIG. 11A. Representative Western blot analysis of FAK phosphorylation (Tyr397) following silencing of MB3 lincRNA in DAOY cells with the siRNAs set forth in SEQ ID NOs: 2-7 compared to control (si-negative).

FIG. 11B. Representative Western blot analysis of FAK phosphorylation (Tyr397) following silencing of MB3 lincRNA in UW-228 cells with the siRNAs set forth in SEQ ID NOs: 2-7 compared to control (si-negative).

FIG. 11C. Graphical representation of relative expression levels of phospho-FAK^{Y397} relative to FAK (ratio of phospho-FAK^{Y397}/FAK) in DAOY cells treated with the siRNAs set forth in SEQ ID NOs: 2-7 compared to control (si-negative).

FIG. 11D. Graphical representation of relative expression levels of phospho-FAK^{Y397} relative to FAK (ratio of phospho-FAK^{Y397}/FAK) in UW-228 cells treated with the siRNAs set forth in SEQ ID NOs: 2-7 compared to control (si-negative).

FIG. 12A. Representative immunofluorescent analysis of phospho-FAK^{Y397} in DAOY and UW-228 cells over-expressing MB3 lincRNA (MB3 linc plasmid), or transfected with an empty plasmid.

FIG. 12B. Graphical representation (n=3) corresponding to FIG. 12A, of relative expression level of phospho-FAK^{Y397} in DAOY and UW-228 cells over-expressing MB3 lincRNA (MB3 lincRNA plasmid), or transfected with an empty plasmid.

FIG. 13A. Representative image of cells immunostained for THSD7A and phospho-FAK(ser 722).

FIG. 13B. Representative image of cells over-expressing MB3 lincRNA, immunostained for THSD7A and phospho-FAK(ser 722).

FIG. 14A. Representative images of tube formation induced by medium of UW-228 cells overexpressing MB3 lincRNA (linc plasmid; right panel), and control cells transfected with an empty plasmid (left panel).

FIG. 14B. Graphical representation corresponding to FIG. 14A, of total tube length per treatment, measured using ImageJ Software.

FIG. 15. Differential expression of MB4 lincRNA (SEQ ID NO: 8) in the four medulloblastoma core groups (n=64).

FIG. 16. NanoString gene expression analysis of the outlier sample of FIG. 15 Group 3.

FIG. 17A. Differential expression of MB4 lincRNA (SEQ ID NO: 8) in the four medulloblastoma core groups from the Kool dataset.

FIG. 17B. Differential expression of MB4 lincRNA (SEQ ID NO: 8) in the four medulloblastoma core groups from the Gilbertson dataset,

FIG. 18. Differential expression of MB4 lincRNA (SEQ ID NO: 8) from publicly available data from microarray studies of normal brain (“N-Brain”), normal cerebellum (“N-Cerebellum”), medulloblastoma (including the Kool and Gilbertson studies) (“M-All”, “M-G3”, “M-G4”, “M-WNT”, “M-SHH”) and brain tumors other than medulloblastoma: glioma, glioblastoma, polycystic astrocytoma (“PA”), ATRT, CNS-PNET and ependymoma (“Ep”) (n=1389).

FIG. 19. Case study: detection of tumor recurrence by MB4 lincRNA liquid biopsy preceding MRI detection of the tumor.

FIG. 20A-B. Setting ddPCR threshold: a manual global threshold was determined from the signals observed in no-template controls (NTCs) and on a set of samples that included tenfold dilutions of the target DNA.

FIG. 21. MB4 lincRNA expression in the plasma pools of 4-12 non-oncology pediatric patients (normal; n=2) and in plasma samples of pediatric cancer patients diagnosed with SHH (n=1), Group 3 (n=4), and pediatric cancer patients diagnosed with Group 4 (n=1).

FIG. 22. MB4 lincRNA expression in the plasma pools of 4-12 non-oncology pediatric patients (normal; n=5) and in plasma samples of random pediatric cancer patients diagnosed with SHH (n=1), pineoblastoma (n=1), Ewing sarcoma (n=1), neuroblastoma (n=1).

### DETAILED DESCRIPTION OF THE INVENTION

The present invention relates to identification and treatment of medulloblastoma using MB3 lincRNA, set forth in SEQ ID NO: 1, MB4 lincRNA, set forth in SEQ ID NO: 8, and inhibitors thereof.

As used herein, the term “MB3 lincRNA” refers to the RNA encoded by the sequence set forth in SEQ ID NO: 1. The terms “MB3 lincRNA” and “lincRNA 13888” are interchangeable.

As used herein, the term “MB4 lincRNA” refers to the RNA encoded by the sequence set forth in SEQ ID NO: 8. Medulloblastoma is defined by four core subgroups, which are termed SHH, WNT, Group 3 and Group 4. Groups SHH and WNT groups are defined as having aberrant Sonic Hedgehog and WNT signaling pathways. Group 3 and Group 4 are collectively referred to as non-WNT/non-SHH medulloblastoma, meaning that these are tumors which are non-WNT-activated (namely, activation of WNT pathway is not involved) and non-SHH activated (namely, activation of SHH pathway is not involved). The currently acceptable classification of medulloblastomas is set forth in the revised 2016 WHO classification (Louis et al., Acta Neuropathol. 131:803-820, 2016). “Metastatic medulloblastoma” as used herein indicates a medulloblastoma predisposed to metastases, either already formed or predisposed to future metastases.

The etiologies of Group 3 and 4, which represent 27% and 34% of MB, respectively, are poorly defined. Group 3 tumors are usually found in infants and children, and almost never observed in adults. Their tumors are frequently metastatic. The WNT subgroup has a very good prognosis. Prognosis for SHH group and for Group 4 tumors is considered intermediate and as the metastasis rates of Group 3 and Group 4, are up to 45% and 40%, respectively—Group 3 carries the poorest prognosis. Recent studies have divided the core groups into several subgroups using genomic analysis.

Long non coding RNA (lincRNA or lncRNA) are RNAs of more than 200 nucleotides in length that lack an open reading frame of significant length. LincRNAs are involved in regulating gene expression at various levels, including chromatin modification, transcription and posttranscriptional processing. lincRNAs were found to be deregulated in several human cancers, and specifically, were shown to be involved in tumorigenesis, tumor cells proliferation, invasion, migration, apoptosis and angiogenesis. Most in-silico studies of lincRNAs rely on RNA-Seq results initially, with quality-control filtering steps to remove reads arising from false background noise.

Early diagnosis of brain tumor is crucial for the best possible clinical practice. In addition, tumor subtype definition in brain cancer is of great importance since many treatments and ultimately, prognosis depend on it. Current methods of brain tumor diagnosis are costly, dangerous to the patient, time-consuming and do not allow for real-time personalized patient care. It is now disclosed herein that MB4 lincRNA serves as a diagnostic liquid biopsy marker for non-WNT/non-SHH medulloblastoma. As exemplified hereinbelow, this lincRNA marker is shown to be useful in detecting the tumor, tumor recurrence and tumor metastasis even preceding MRI detection, providing essential means for molecular disease characterization, and assessment of tumor response to treatment.

In primary brain tumors, the concept of “integrated diagnosis” based upon both morphological and molecular findings is used to distinguish between entities with overlapping histological features. Precise molecular diagnosis and classification of specific tumor subtypes will result in the implementation of precision medicine for brain cancer. Molecular markers help stratify subgroups with disparate outcome, allowing for milder treatments in less aggressive diseases. Moreover, assessing tumor response to treatment nowadays rests heavily on neuroimaging which is lacking the ability to differentiate between actual tumor progression and edema. Molecular repetitive longitudinal testing could help objectively assess the tumor residual burden during and post treatment. Significantly, in brain tumors, the current procedures for tumor biopsy are invasive, expensive and carry additional risks to patients. Moreover, tissue biopsies reflect only a small and localized region of the tumor and can fail to be representative of the intra-tumoral heterogeneity or of its evolutions over time. Molecular profiling of a brain neoplasm is possible by analyzing circulating components of the tumor in liquid biopsies such as blood. Thus, a simple blood draw could be used to monitor tumor evolution in real time and reduce the need for surgical biopsies for patient care.

In some embodiments, there is provided herein a method for classifying a medulloblastoma tumor of a subject, the method comprising measuring a level of at least one lincRNA selected from the group consisting of MB3 lincRNA (SEQ ID NO: 1) and MB4 lincRNA (SEQ ID NO: 8) in a sample of the medulloblastoma tumor, wherein a level of MB3 lincRNA above a predefined threshold is indicative of Group 3 medulloblastoma; and wherein a level of MB4 above a predefined threshold is indicative of non-WNT/non-SHH medulloblastoma.

In some embodiments, there is provided herein a method for classifying a medulloblastoma tumor of a subject, the method comprising measuring a level of at least one lincRNA selected from the group consisting of MB3 lincRNA (SEQ ID NO: 1) and MB4 lincRNA (SEQ ID NO: 8) in a sample of the medulloblastoma tumor, wherein a level of MB3 lincRNA above a predefined threshold is indicative of metastatic medulloblastoma; and wherein a level of MB4 above a predefined threshold is indicative of non-WNT/non-SHH medulloblastoma.

In some embodiments, a method for treating a subject with a cancerous tumor over-expressing MB3 lincRNA is provided, the method comprising administering an inhibitory nucleic acid targeting MB3 lincRNA to the subject.

In some embodiments, the method further comprises measuring a level of MB3 lincRNA in a sample of the cancerous tumor and determining whether the level of MB3 lincRNA is above a predefined threshold.

In some embodiments, a method for treating a subject with a cancerous tumor over-expressing MB4 lincRNA is provided, the method comprising administering an inhibitory nucleic acid targeting MB4 lincRNA to the subject

In some embodiments, the method further comprises measuring a level of MB4 lincRNA in a sample of the cancerous tumor and determining whether the level of MB4 lincRNA is above a predefined threshold.

In some embodiments, the tumor is a medulloblastoma.

In some embodiments, there is provided a method for reducing or inhibiting migration and invasion of cells, the method comprising contacting the cells with an MB3 lincRNA inhibitor. In some embodiments, the cells are medulloblastoma cells over-expressing MB3 lincRNA. In some embodiments, the contacting is carried out in vivo. In other embodiments, the contacting is carried out ex vivo.

In some embodiments, there is provided a method for reducing or inhibiting migration and invasion of medulloblastoma cells, the method comprising contacting the medulloblastoma cells with an MB3 lincRNA inhibitor. In some embodiments, the medulloblastoma cells are medulloblastoma cells over-expressing MB3 lincRNA. In some embodiments, the contacting is carried out in vivo. In other embodiments, the contacting is carried out ex vivo.

In some embodiments, there is provided a method for reducing or inhibiting migration and invasion of cells, the method comprising contacting the cells with an MB4 lincRNA inhibitor. In some embodiments, the cells are medulloblastoma cells over-expressing MB4 lincRNA. In some embodiments, the contacting is carried out in vivo. In other embodiments, the contacting is carried out ex vivo.

In some embodiments, there is provided a method for reducing or inhibiting migration and invasion of medulloblastoma cells, the method comprising contacting the medulloblastoma cells with an MB4 lincRNA inhibitor. In some embodiments, the medulloblastoma cells are medulloblastoma cells over-expressing MB4 lincRNA. In some embodiments, the contacting is carried out in vivo. In other embodiments, the contacting is carried out ex vivo.

In some embodiments, there is provided a nucleic acid targeting MB3 lincRNA, for use in the treatment of medulloblastoma. In some embodiments, there is provided herein a pharmaceutical composition comprising a nucleic acid targeting MB3 lincRNA, for use in the treatment of medulloblastoma. In some embodiments, the nucleic acid or the pharmaceutical composition is for use in the treatment of medulloblastoma characterized by expression of MB3 lincRNA above a predefined threshold level. In some embodiments, the nucleic acid or the pharmaceutical composition is for use in the treatment of Group 3 medulloblastoma. In some embodiments, the nucleic acid or the pharmaceutical composition is for use in the treatment of metastatic medulloblastoma. In some embodiments the nucleic acid is an inhibitory nucleic acid.

In some embodiments, there is provided a nucleic acid targeting MB4 lincRNA, for use in the treatment of medulloblastoma. In some embodiments, there is provided herein a pharmaceutical composition comprising a nucleic acid targeting MB4 lincRNA, for use in the treatment of medulloblastoma. In some embodiments, the nucleic acid or the pharmaceutical composition is for use in the treatment of medulloblastoma characterized by expression of MB4 lincRNA above a predefined threshold level. In some embodiments, the inhibitory nucleic acid or the pharmaceutical composition is for use in the treatment of Group 4 medulloblastoma. In some embodiments, the nucleic acid or the pharmaceutical composition is for use in the treatment of non-WNT/non-SHH medulloblastoma. In some embodiment the nucleic acid is an inhibitory nucleic acid.

In some embodiments, there is provided herein a method for characterizing a medulloblastoma tumor sample, particularly profiling RNA expression in a medulloblastoma tumor sample, the method comprising:

measuring a level of at least one lincRNA selected from the group consisting of MB3 lincRNA (SEQ ID NO: 1) and MB4 lincRNA (SEQ ID NO: 8) in the medulloblastoma tumor sample;

determining whether the MB3 lincRNA level is above a threshold level indicative of at least one of Group 3 medulloblastoma and metastatic medulloblastoma; and

determining whether the MB4 level is a above a threshold level indicative of non-WNT/non-SHH medulloblastoma,

thereby profiling RNA expression in the medulloblastoma tumor sample.

In some embodiments, there is provided herein a method for characterizing a medulloblastoma tumor sample, particularly profiling RNA expression in a medulloblastoma tumor sample, the method comprising:

measuring a level of at least one lincRNA selected from the group consisting of MB3 lincRNA (SEQ ID NO: 1) and MB4 lincRNA (SEQ ID NO: 8) in the medulloblastoma tumor sample;

detecting an MB3 lincRNA level above a threshold level indicative of at least one of Group 3 medulloblastoma and metastatic medulloblastoma; and/or

detecting an MB4 level above a threshold level indicative of non-WNT/non-SHH medulloblastoma,

thereby profiling RNA expression in the medulloblastoma tumor sample.

In some embodiments, there is provided herein a method for characterizing a biological fluid sample, particularly profiling RNA expression in a biological fluid sample, the method comprising:

measuring a level of at least one lincRNA selected from the group consisting of MB3 lincRNA (SEQ ID NO: 1) and MB4 lincRNA (SEQ ID NO: 8) in the biological sample; and

determining whether the MB4 level is a above a threshold level indicative of the presence of non-WNT/non-SHH medulloblastoma tumor in the subject.

In some embodiments, there is provided herein a method for identifying predisposition to metastasis of cancerous tumors using the MB3 lincRNA as disclosed herein.

The methods of the invention can be performed in vitro, ex vivo or in vivo.

In some embodiments, there is provided herein a synthetic double-stranded RNA targeting MB3 lincRNA or a vector encoding the same, selected from the group consisting of:

(A) a dsRNA comprising a sense strand comprising the sequence set forth in SEQ ID NO: 2 or a sequence with at least 90% identity to the sequence set forth in SEQ ID NO: 2, and an antisense strand comprising the sequence set forth in SEQ ID NO: 3 or a sequence with at least 90% identity to the sequence set forth in SEQ ID NO: 3;

(B) a dsRNA comprising a sense strand comprising the sequence set forth in SEQ ID NO: 4 or a sequence with at least 90% identity to the sequence set forth in SEQ ID NO: 4, and an antisense strand comprising the sequence set forth in SEQ ID NO: 5 or a sequence with at least 90% identity to the sequence set forth in SEQ ID NO: 5; and

(C) a dsRNA comprising a sense strand comprising the sequence set forth in SEQ ID NO: 6 or a sequence with at least 90% identity to the sequence set forth in SEQ ID NO: 6, and an antisense strand comprising the sequence set forth in SEQ ID NO: 7 or a sequence with at least 90% identity to the sequence set forth in SEQ ID NO: 7,

wherein each strand is between 19 to 25 nucleotides in length.

In some embodiments, each strand is between 15 to 25 nucleotides in length, for example 15, 16, 17, 18, 19, 20, 21, 22, 23, 24 or 25 nucleotides in length. Each possibility represents a separate embodiment of the present invention. In some embodiments, each strand is 16-25 nucleotides in length, 17-25 nucleotides in length, 18-25 nucleotides in length, 19-25 nucleotides in length, 15-21 nucleotides in length, 16-21 nucleotides in length, 17-21 nucleotides in length, 18-21 nucleotides in length, 19-21 nucleotides in length. Each possibility represents a separate embodiment of the present invention.

In some embodiments, there is provided herein a synthetic antisense oligonucleotide targeting MB4 lincRNA, selected from the group consisting of:

(A) an antisense oligonucleotide comprising the sequence set forth in SEQ ID NO: 9, or a sequence with at least 90% identity to the sequence set forth in SEQ ID NO: 9;

(B) an antisense oligonucleotide comprising the sequence set forth in SEQ ID NO: 10, or a sequence with at least 90% identity to the sequence set forth in SEQ ID NO: 10; and

(C) an antisense oligonucleotide comprising the sequence set forth in SEQ ID NO: 11, or a sequence with at least 90% identity to the sequence set forth in SEQ ID NO: 11,

wherein the antisense oligonucleotide is between 10 to 30 nucleotides in length.

In some embodiments, the antisense oligonucleotide is between 15-25 nucleotides in length, for example, 15, 16, 17, 18, 19, 20, 21, 22, 23, 24, 25 nucleotides in length. Each possibility represents a separate embodiment of the present invention.

In some embodiments, the antisense oligonucleotide is 15-25 nucleotides in length, 16-25 nucleotides in length, 17-25 nucleotides in length, 18-25 nucleotides in length, 19-25 nucleotides in length, 20-25 nucleotides in length, 15-20 nucleotides in length, 16-20 nucleotides in length, 17-20 nucleotides in length, 18-20 nucleotides in length, 19-20 nucleotides in length or 20 nucleotides in length. Each possibility represents a separate embodiment of the present invention.

An antisense oligonucleotide as disclosed herein typically comprises phosphorothioate bonds.

An antisense oligonucleotide as disclosed herein typically comprises modified bases at each end of the sequence, such as modified RNA bases, for example: 2′-O-Me RNA base. In some embodiments, the antisense oligonucleotide comprises 1-5 modified bases at each end of the sequence, for example, 1, 2, 3, 4, 5 modified bases at each end of the sequence. Each possibility represents a separate embodiment of the present invention.

In some embodiments, there is provided a synthetic antisense oligonucleotide selected from the group consisting of: ASO1 (SEQ ID NO: 9); ASO2 (SEQ ID NO: 10); and ASO3 (SEQ ID NO: 11). Each possibility represents a separate embodiment of the present invention.

In some embodiments, there is provided a method for treating medulloblastoma as disclosed herein, by administering a combination of, or a mixture of, the inhibitory nucleic acids disclosed herein.

The therapeutic nucleic acids provided in the present disclosure may be administered systemically or locally, for example adjacent to the tumor or cavity from which the tumor was resected.

In some embodiments, vectors encoding for, or carrying, the nucleic acids provided herein are used.

The term “construct”, as used herein, refers to an artificially assembled or isolated nucleic acid molecule which may include one or more nucleic acid sequences, wherein the nucleic acid sequences may include coding sequences (that is, sequence which encodes an end product), regulatory sequences, non-coding sequences, or any combination thereof. The term construct includes, for example, vector but should not be seen as being limited thereto.

As used herein the term “vector” refers to recombinant constructs engineered to encode or express polynucleotides in a target cells, such as DNA, RNA, miRNA, shRNA, siRNA, antisense oligonucleotides, and the like. Vectors may include such vectors as, but not limited to, viral and non-viral vectors, plasmids, and the like.

In some embodiments, identify to a selected nucleic acid sequence of the present invention is at least 90%, at least 95% or 100% identity. Each possibility represents a separate embodiment.

In some embodiments, there is provided a method for the treatment of group 3 medulloblastoma in a subject comprising administering to a subject having group 3 medulloblastoma a pharmaceutical composition comprising an effective amount of an inhibitor of the expression, level or activity of MB3 lincRNA, set forth in SEQ ID NO: 1.

According to some embodiments, the activity of MB3 lincRNA is regulation of Thrombospondin type 1 domain containing 7A (THSD7A), which encodes a protein shown to be involved in cell migration, tumor invasion, endothelial blood tube formation (angiogenesis) and in metastasis in some diseases. THSD7A is a membrane-associated N-glycoprotein with a soluble form. THSD7A expression status was linked to vascular invasion, nodal stage and the presence of distant metastases in different types of cancers. In esophageal squamous cell carcinoma (ESCC) it was shown that THSD7A has a role in tumor development and could promote the proliferating, migrating as well as invading processes of tumors. As exemplified hereinbelow, silencing MB3 lincRNA associates with reduction in THSD7A expression, while overexpression of MB3 lincRNA associates with an increase in THSD7A expression. In some embodiments, there is provided herein a method for reducing THSD7A expression in cells, for example in cancer cells such as medulloblastoma cells, by contacting the cells with an MB3 lincRNA inhibitor.

Soluble THSD7A is the functional form that promotes endothelial cell migration and significantly increases the phosphorylation level of Focal adhesion kinase (FAK). FAK is a cytoplasmic tyrosine kinase which is part of the cell-ECM adhesion structure called focal adhesion. Increased expression and/or activation of FAK are found in a variety of human cancers. After FAK is activated by phosphorylation, vinculin is recruited to FAK modulating cell migration and actin polymerization. FAK autophosphorylation at Y397 and its exposure for binding Src family kinases, phosphorylates additional sites on FAK leading to its full activation. As exemplified hereinbelow, silencing MB3 lincRNA associates with reduction in FAK phosphorylation, while overexpression of MB3 lincRNA increases FAK phosphorylation.

Provided herein is an inhibitor of MB3 lincRNA which is any one of an inhibitor of cancer, an inhibitor of cell migration, an inhibitor of cell proliferation, an inhibitor of FAK phosphorylation, and an inhibitor of cell invasion.

In some embodiments, there is provided a method for the treatment of group 4 medulloblastoma in a subject in need thereof, the method comprising administering to a subject having group 4 medulloblastoma a pharmaceutical composition comprising an effective amount of an inhibitor of the expression, level or activity of MB4 lincRNA, set forth in SEQ ID NO: 8.

In some embodiments, there is provided a method of treating group 4 medulloblastoma in a subject in need thereof, comprising isolating at least one lincRNA transcript in a biological sample from a subject having, or suspected of having, medulloblastoma, wherein the at least one isolated lincRNA transcript is MB4 lincRNA having a nucleotide sequence set forth in SEQ ID NO:1; determining that the level of the at least one isolated lincRNA transcript is above a control level, thereby diagnosing the subject as having group 4 medulloblastoma; and treating the subject for group 4 medulloblastoma.

The term “inhibitor” as used herein refers to any inhibitor capable of inhibiting, reducing or suppressing the activity or expression of lincRNA. An inhibitor can be a nucleic acid, single or double stranded. An inhibitor includes, but is not limited to, a gapmer, an shRNA, an asRNA, an siRNA, a CRISPR, a TALEN, or a Zinc-finger nuclease. In some embodiments, the inhibitor is a small molecule. In some embodiments, the inhibitor selectively inhibits migration and invasion in tumor cells.

As used herein a nucleic acid targeting a sequence (e.g., targeting MB3 lincRNA or MB4 lincRNA) refers to a nucleic acid molecule which specifically recognizes the target sequence and it is capable of functionally inhibiting said sequence. A nucleic acid targeting a sequence may hybridize to said sequence under stringent conditions. As used herein an “inhibitory nucleic acid” targeting a particular sequence (e.g., targeting MB3 lincRNA or MB4 lincRNA) refers to a nucleic acid molecule which is capable of sequence-specific inhibition of the target sequence. An inhibitory nucleic acid targeting a particular sequence contains a sequence that is complementary to, or capable of hybridizing to, a subsequence within the target sequence. The inhibitory nucleic acid typically facilitates degradation of the target sequence, thereby inhibiting expression and/or activity of the target sequence.

As used herein, “reducing the level of” a gene, “inhibiting expression” and the like, includes a decrease in the level of RNA encoded by the gene in the cell or organism. As used herein, “reducing” or “reduction” includes at least a partial reduction of the level of an agent (such as lincRNA), which means that the level is reduced at least 25%, preferably at least 50%, relative to a cell or organism lacking the inhibitory agent as disclosed herein.

The reduction can be determined by methods with which the skilled worker is familiar. Thus, the reduction of level of the lincRNA can be determined for example by biochemical techniques such as Northern hybridization, nuclease protection assay, reverse transcription (quantitative RT-PCR), digital PCR, droplet digital PCR, and the like.

In some embodiments, the inhibitor is an interfering RNA (RNAi).

As used herein, the term “RNAi” refers to any type of interfering RNA, including but are not limited to, siRNAi, shRNAi, stRNAi, endogenous microRNA and artificial microRNA.

In some embodiments, the inhibitor is an siRNA.

The term “siRNA” also refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 10-50 nucleotides in length (e.g., each complementary sequence of the double stranded siRNA is about 10-22 nucleotides in length, and the double stranded siRNA is about 10-22 base pairs in length, preferably about 19-22 base nucleotides, preferably about 17-19 nucleotides in length, e.g., 10, 11, 12, 13, 14, 15, 16, 17, 18, 19, 20, 21, or 22 nucleotides in length). As used herein “shRNA” or “small hairpin RNA” (also called stem loop) is a type of siRNA. In one embodiment, these shRNAs are composed of a short, e.g. about 10 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

In some embodiments, the MB3 lincRNA inhibitor comprises at least one siRNA selected from the group consisting of: siRNA1 (SEQ ID NOs:2-3); siRNA2 (SEQ ID NOs:4-5); and siRNA3 (SEQ ID NOs:6-7). Each possibility represents a separate embodiment of the present invention.

In some embodiments, the MB3 lincRNA inhibitor comprises a plurality of siRNAs selected from the group consisting of: siRNA1 (SEQ ID NOs:2-3); siRNA2 (SEQ ID NOs:4-5); and siRNA3 (SEQ ID NOs:6-7). Each possibility represents a separate embodiment of the present invention.

In some embodiments, the MB3 lincRNA inhibitor comprises siRNA1 (SEQ ID NOs:2-3); siRNA2 (SEQ ID NOs:4-5); and siRNA3 (SEQ ID NOs:6-7). In some embodiments, the inhibitor is consisting of siRNA1 (SEQ ID NOs:2-3); siRNA2 (SEQ ID NOs:4-5); and siRNA3 (SEQ ID NOs:6-7).

In some embodiments, the MB4 lincRNA inhibitor is single stranded nucleic acid. In some embodiments, the MB4 lincRNA inhibitor is an antisense oligonucleotide.

Antisense oligonucleotide, as used herein, refers to an oligonucleotide that is substantially or 100% complementary to a target sequence of interest. It may also include the antisense region of both oligonucleotides that are formed from two separate strands, as well as unimolecular oligonucleotides that are capable of forming hairpin or dumbbell type structures. By “target sequence” is meant any nucleic acid sequence whose expression or activity is to be modulated. The target nucleic acid can be DNA or RNA, such as MB4 lincRNA.

The terms “antisense oligonucleotides” or “ASOs” are interchangeable and refer to antisense oligonucleotides that bind a target RNA and trigger degradation thereof by an enzyme that cleaves the RNA strand in a DNA/RNA heteroduplex (endogenous RNase H). Unmodified (naïve) DNA oligonucleotides are rapidly degraded in serum and the intracellular environment and thus have poor efficacy. DNA oligonucleotides with a phosphorothioate (PS) backbone improve the functionality of ASOs by substantial nuclease resistance yet retaining the ability to trigger RNase H. PS modification decreases the melting temperature (Tm) of the oligonucleotide, which can lower potency. Thus, 2-O-Methyl (2_OMe) RNA is added to the sequence of the oligonucleotide, in order to enhance the Tm of the resulting antisense oligonucleotide.

In some embodiments, the MB4 lincRNA inhibitor is at least one antisense oligonucleotide selected from the group consisting of: ASO1 (SEQ ID NO: 9); ASO2 (SEQ ID NO: 10); and ASO3 (SEQ ID NO: 11). Each possibility represents a separate embodiment of the present invention.

In some embodiments, the MB4 lincRNA inhibitor comprises a plurality of said at least one antisense oligonucleotide.

In some embodiments, the MB4 lincRNA inhibitor comprises ASO1 (SEQ ID NO: 9); ASO2 (SEQ ID NO: 10); and ASO3 (SEQ ID NO: 11).

In some embodiments, the MB4 lincRNA inhibitor is consisting of ASO1 (SEQ ID NO: 9); ASO2 (SEQ ID NO: 10); and ASO3 (SEQ ID NO: 11).

The term “plurality” as used herein refers to ‘at least two’ or ‘two or more’.

As used herein, the terms “treat,” “treating,” “treatment,” and the like refer to reducing or ameliorating MB and/or a symptom associated therewith. It will be appreciated that, although not precluded, treating does not require complete elimination of MB. Treating may include a health care professional or diagnostic scientist making a recommendation to a subject for a desired course of action or treatment regimen, e.g., a prescription.

In some embodiments, treating comprises any one or more of inhibiting tumor growth, suppressing tumor growth, reducing incidence of metastases and inhibiting formation of metastases.

Thus, treating as disclosed herein is particularly beneficial as it is directed to the growth and metastasis of metastatic medulloblastoma and other metastatic cancers.

In some embodiments, the method disclosed herein further comprises, prior to said administering, the step of: determining the expression of MB3 lincRNA, set forth in SEQ ID NO:1, in a biological sample derived from a subject, wherein expression of MB3 lincRNA above a predetermined threshold indicates that said subject is having at least one of group 3 medulloblastoma and metastatic medulloblastoma.

In some embodiments, the method disclosed herein further comprises, prior to said administering, the step of: determining the expression of MB4 lincRNA, set forth in SEQ ID NO: 8, in a biological sample derived from a subject, wherein expression of MB4 lincRNA above a predetermined threshold indicates that said subject is having non-WNT/non-SHH medulloblastoma.

In some embodiments, determining the expression of MB3 lincRNA, MB4 lincRNA or both is performed by microarray. Single strands of complementary DNA for MB3 lincRNA and/or MB4 lincRNA or for fragments thereof, also termed hereinafter ‘probes’, are immobilized on a grid (array). RNA is extracted from a sample of interest, converted to complimentary DNA, labeled and hybridized to the array. Measuring the quantity of label on each spot in the array then yields an intensity value that correlates to the abundance of the corresponding RNA transcript in the sample. Commonly, labeling is fluorescent. Typically, expression levels are normalized. Quantification can utilize a laser scanner that determines the intensities of each label over the entire array. To judge or determine the significance of the conclusions derived through quantification, replicates may be used.

In preparing the probes, the various DNA fragments may be manipulated, for example, so as to provide for the DNA sequences in the proper orientation. Toward this end, adapters or linkers may be employed or other manipulations may be involved.

In some embodiments, the expression level of the tested sample (also termed ‘test level’) is compared to a standard level or a control level.

In some embodiments, the control level or the standard level is a reference value derived from a healthy individual or a population of healthy individuals. “Healthy” typically include individuals not afflicted with medulloblastoma and preferably not afflicted with any other malignancy or brain disease. In some embodiments, the reference value is similar or identical to said predetermined threshold.

The term “healthy” as used herein is interchangeable with “reference”, “normal” and “control”. This term refers to MB3 lincRNA level/MB4 lincRNA measured in normal individuals. A “healthy” or “normal” individual is defined herein as an individual without detectable medulloblastoma or other tumor or symptoms thereof, determined by conventional diagnostic methods. The level corresponding to a healthy individual may be obtained from a single individual, a plurality of individuals, a population, database or it may be a statistic value. The control level or the standard level may be referred to as “non-medulloblastoma level”.

In some embodiments, determining the expression of MB3 lincRNA and/or MB4 lincRNA is performed by applying RNAseq. This approach provides a broad picture of the genes that are transcribed in the patient at once. It enables selecting the gene(s) of interest within massive data.

In some embodiments, the method comprises, prior to said administering, the step of: determining the expression of MB3 lincRNA, set forth in SEQ ID NO:1, in a biological sample derived from a subject, wherein if the level of expression of MB3 lincRNA is increased relative to the control, or the reference, or the predetermined threshold then said subject is having metastatic tumor.

In some embodiments, determining the expression of MB3 lincRNA and/or MB4 lincRNA is performed by a quantitative amplification method. In some embodiments, the quantitative amplification method is quantitative real-time PCR carried out by extracting RNA from the sample, reverse-transcribing the RNA to cDNA, and subjecting the cDNA to real-time PCR using primers and probes specific for MB3 lincRNA and/or MB4 lincRNA.

In additional embodiments, the quantitative amplification method is droplet-digital PCR (ddPCR), which is a quantitative PCR method based on water-oil emulsion droplet technology. A sample is fractionated into ˜20,000 droplets, and PCR amplification of the template molecules occurs in each individual droplet. The reaction area is a droplet that is formed in a water-oil emulsion. Techniques for performing droplet digital PCR and emulsion droplet digital PCR are available and include, but are not limited to, those described in Hindson et al., Anal Chem, 83:8604-8610 (2011); Pinheiro et al., Anal Chem, 84: 1003-1011 (2012); and Jones et al., Virohgicai Methods, 202: 46-53 (2014). Droplet digital PCR systems and emulsion droplet digital PCR systems also are commercially available from sources such as, for example, the QX200™ DROPLET DIGITAL™ PCR system (Bio-Rad Laboratories, Inc., Hercules, Calif.).

In some embodiments, expression above a predetermined threshold refers to expression that is at least 50% higher than the control expression level, for example, at least 50% higher than the expression level of the biomarker in a healthy individual or a population of healthy individuals.

In some embodiments, expression above a predetermined threshold refers to expression that is at least 50% higher than the control expression level.

In some embodiments, expression above a predetermined threshold refers to expression that is at least 60% higher than the control expression level.

In some embodiments, expression above a predetermined threshold refers to expression that is at least 70% higher than the control expression level.

In some embodiments, expression above a predetermined threshold refers to expression that is at least 80% higher than the control expression level.

In some embodiments, expression above a predetermined threshold refers to expression that is at least 90% higher than the control expression level

In some embodiments, expression above a predetermined threshold refers to expression that is at least 100% (twice) higher than the control expression level

In some embodiments, expression above a predetermined threshold refers to expression that is at least twice higher than the control expression level.

In some particular embodiments, expression above 10 copies per 0.5 ml plasma using droplet digital PCR threshold refers to expression that is higher than the control expression level.

As used herein, a “subject” is any organism or animal to whom which treatment or prophylaxis treatment is desired. Such animals include mammals, preferably a human. The term “subject” also refers to any living organism from which a biological sample can be obtained. The term includes, but is not limited to, humans. The term does not denote a particular age or gender. Thus, adult and newborn subjects, as well as fetuses, whether male or female, are intended to be covered. The term “subject” is also intended to include transgenic species. In another embodiment, the subject is an experimental animal or animal substitute as a disease model.

The term “sample” as used herein refers to a cell or population of cells or a quantity of tissue or fluid from a subject. Most often, the sample has been removed from a subject, but the term “sample” can also refer to cells or tissue analyzed in vivo, i.e. without removal from the subject. Often, a “sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), tumor biopsy and or whole blood, plasma, serum, urine, saliva, cell culture, or cerebrospinal fluid (CSF). Biological samples also include tissue biopsies, cell culture. A biological sample or tissue sample can refer to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture or constituents thereof, such as, culture media. In some embodiments, the sample is from a resection, biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo.

The term “tissue” is intended to include intact cells, blood, blood preparations such as plasma and serum, bones, joints, muscles, smooth muscles, and organs.

In some embodiments, the biological sample comprises tumor biopsy and or whole blood.

In some embodiment, the treatment methods disclosed herein are combined with any one or more of radiation therapy, chemotherapy and biological therapy. Radiation therapy and chemotherapy can be applied in parallel, prior to and/after the method disclosed herein. In some embodiment, the method disclosed herein is combined with bone marrow transplantation. In some embodiments, bone marrow transplantation precedes the method disclosed herein. In some embodiments, bone marrow transplantation is carried out after applying the method disclosed herein. In some embodiments, the method disclosed herein is carried out prior to and following bone marrow transplantation.

In some embodiment, the method disclosed herein is combined with tumor resection. In some embodiments, tumor resection precedes the method disclosed herein. In some embodiments, tumor resection is carried out after applying the method disclosed herein. In some embodiments, the method disclosed herein is carried out prior to and following tumor resection.

In some embodiments, the pharmaceutical composition further comprises a pharmaceutical acceptable carrier.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

The term also encompasses any of the agents approved by a regulatory agency of the US Federal government or listed in the US Pharmacopeia for use in animals, including humans.

In some embodiments, there is provided a method for detecting group 3 medulloblastoma in a biological sample, the method comprising: determining the expression of MB3 lincRNA, set forth in SEQ ID NO:1, in the biological sample,

wherein expression of MB3 lincRNA above a predetermined threshold indicates the presence of group 3 medulloblastoma.

In some embodiments, there is provided a method for detecting group 3 medulloblastoma in a biological sample, the method comprising: determining the expression level of MB3 lincRNA, set forth in SEQ ID NO:1, in the biological sample, comparing the expression level of determining the expression level of MB3 lincRNA to a reference level,

wherein the reference level is the level of MB3 lincRNA in a healthy population, and

wherein expression of MB3 lincRNA above the reference level indicates the presence of group 3 medulloblastoma.

In some embodiments, there is provided a method for detecting group 4 medulloblastoma in a biological sample, the method comprising: determining the expression of MB4 lincRNA, set forth in SEQ ID NO: 1, in the biological sample, wherein expression of MB4 lincRNA above a predetermined threshold indicates the presence of group 4 medulloblastoma.

In some embodiments, there is provided a method for detecting group 4 medulloblastoma in a biological sample of a subject, the method comprising: determining the expression level of MB4 lincRNA, set forth in SEQ ID NO: 1, in the biological sample, comparing the expression level of MB4 lincRNA to a reference level, wherein the reference level is the level of MB4 lincRNA in a healthy population, and wherein expression of MB4 lincRNA above the reference level indicates the presence of group 4 medulloblastoma.

The terms “reference level” and “control level” as used herein, are interchangeable.

In some embodiments, determining the expression level of MB3 lincRNA, set forth in SEQ ID NO:1, and/or MB4 lincRNA, set forth in SEQ ID NO: 8, is carried out using microarray assay.

In some embodiments, detecting the presence of MB3 lincRNA or the level thereof, and/or MB4 lincRNA or the level thereof, is performed by microarray analysis. In some embodiments, the microarray assay comprises a probe set, an array on which the probes are immobilized at specific locations, a sample containing labeled molecules that can bind to the probes, and a detector that is able to measure the spatially resolved distribution of the label after it has bound to the array.

In some embodiments, the probe set comprising a plurality of probes, wherein the plurality of probes comprises (i) a sequence that hybridizes to at least a portion of SEQ ID NO: 1; or (ii) a sequence that is identical to at least a portion of SEQ ID NO: 1.

In some embodiments, the probe set comprising a plurality of probes, wherein the plurality of probes comprises (i) a sequence that hybridizes to at least a portion of SEQ ID NO: 8; or (ii) a sequence that is identical to at least a portion of SEQ ID NO: 8.

In some embodiments, the method further comprises applying a computer model or algorithm for analyzing the expression level/or expression profile of MB3 lincRNA and/or MB4 lincRNA.

In some embodiments, there is provided use of an inhibitor of the expression or activity of MB3 lincRNA, set forth in SEQ ID NO: 1, for the treatment of group 3 medulloblastoma.

In some embodiments, there is provided use of an inhibitor of the expression or activity of MB3 lincRNA, set forth in SEQ ID NO: 1, for the treatment of metastatic medulloblastoma.

In some embodiments, there is provided use of an inhibitor of the expression or activity of MB4 lincRNA, set forth in SEQ ID NO: 8, for the treatment of group 4 medulloblastoma.

In some embodiments, there is provided use of an inhibitor of the expression or activity of MB4 lincRNA, set forth in SEQ ID NO: 8, for the treatment of non-WNT/non-SHH medulloblastoma.

In some embodiments, there is provided a kit for the treatment of at least one of group 3 medulloblastoma and metastatic medulloblastoma comprising a pharmaceutical composition comprising at least one inhibitor of the expression or activity of MB3 lincRNA, set forth in SEQ ID NO: 1.

In some embodiments, there is provided a kit for the treatment of non-WNT/non-SHH medulloblastoma comprising a pharmaceutical composition comprising at least one inhibitor of the expression or activity of MB4 lincRNA, set forth in SEQ ID NO: 8.

In some embodiments, the inhibitor is an RNAi. In some embodiments, the at least one inhibitor is an siRNA selected from the group consisting of: siRNA1 (SEQ ID NOs:2-3); siRNA2 (SEQ ID NOs:4-5); and siRNA3 (SEQ ID NOs:6-7). In some embodiments, the at least one inhibitor comprises a plurality of siRNAs selected from the group consisting of: siRNA1 (SEQ ID NOs:2-3); siRNA2 (SEQ ID NOs:4-5); and siRNA3 (SEQ ID NOs:6-7).

In some embodiments, the at least one inhibitor comprises siRNA1 (SEQ ID NOs:2-3); siRNA2 (SEQ ID NOs:4-5); and siRNA3 (SEQ ID NOs:6-7).

In some embodiments, there is provided a kit for the detection of group 3 medulloblastoma comprising at least one nucleotide probe having a nucleotide sequence complementary to the SEQ ID NO: 1 or fragments thereof.

In some embodiments, there is provided a kit for the detection of metastatic medulloblastoma comprising at least one nucleotide probe having a nucleotide sequence complementary to the SEQ ID NO: 1 or fragments thereof.

In some embodiments, there is provided a kit for the detection of non-WNT/non-SHH medulloblastoma comprising at least one nucleotide probe having a nucleotide sequence complementary to the SEQ ID NO: 8 or fragments thereof.

In some embodiments, there is provided a kit for the detection of group 4 medulloblastoma comprising at least one nucleotide probe having a nucleotide sequence complementary to the SEQ ID NO: 8 or fragments thereof.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1—Expression of MB3 lincRNA in Medulloblastoma Tumor Samples

Dataset EGAD00001003279 containing RNA data from 61 medulloblastoma patient samples was downloaded from the European Genome—phenome Archive (EGA), together with information on the four core medulloblastoma molecular groups: WNT, SHH, medulloblastoma group 3 and medulloblastoma group 4, and information about metastases (metastatic or non-metastatic) at the time of sampling. The RNA and human genome (ucsc hg19) were aligned using Bowtie 2 program. Non-unique alignments were removed and the number of RNA fragments falling on lincRNAs were counted.

RNAseq results normalization: results were normalized using reads per kilobase transcript per million reads (RPKM), where RPKM is a length normalization and is calculated as follows:

RPKM=[no. of counts/(total count×transcript length|)]×10⁹

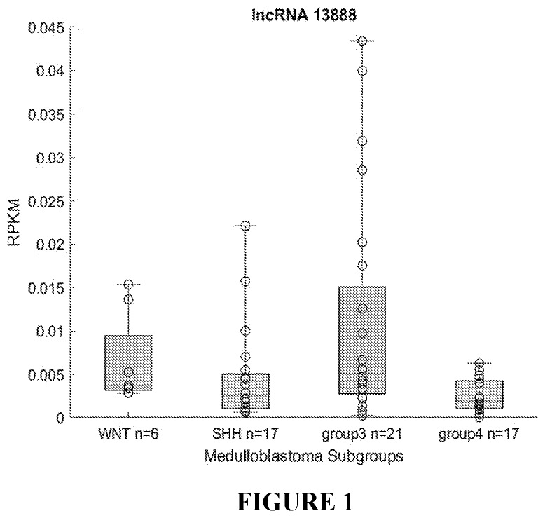

The normalization analysis produced an unbiased estimate of the mean of the genes' expression (FIG. 1). The analysis revealed that the expression of the lincRNA set forth as SEQ ID NO: 1 (Table 1), also referred to herein as “MB3 lincRNA” and “lincRNA 13888” is higher in medulloblastoma group 3, compared to group 4, WNT and SHH (p<0.05 by Kruskal-Wallis). The analysis further showed correlation of MB3 lincRNA over-expression with metastases. Of the patients that expressed high levels of MB3 lincRNA, 50% were metastatic at the time of sampling. On the other hand, approximately 75% of the patients that had low expression of MB3 lincRNA were non-metastatic at the time of sampling.

It is noted that SEQ ID NO: 1 is a DNA sequence, and it is to be understood that the corresponding RNA sequence includes U nucleotides instead of the T nucleotides.

TABLE 1A
Sequences

| SEQ. | | |
|---|---|---|
| ID. | | |
| No. | Name | Sequence** |
| 1 | MB3 lincRNA | cctggacctagaaacaaaaaatgaaagtgga |
| | (natural) | gaaaaccaaacaagaatctttctttcttaaagta |
| | transcript name: | aacatcggactgacatctectttcaaacattcatt |
| | TCONS_00013888 | gaggatctactctgtgagaagaagcaatac |
| | Location (hg19): | [symbol] ccag |
| | chr7:93908311- | ttctagttgacttgattgtcaaacgactgcta |
| | 94021491 | agaaggtgaaatagaggagatgacatga |
| | | atgagggactacattggaatgaagaaga |
| | | acctggaggaggtaaaaaaactgaagg |
| | | ctccggatgtcggacgatggaaaataaa |
| | | aacaacaagcaagtgaaactccctgact |
| | | tatcaaccttactcaagaattccttct |
| 2 | siRNA1 | gacuuaucaaccuuacuca[dt][dt]* |
| | L138SiRNA322 | |
| 3 | siRNA1 | ugaguaagguugauaaguc[dt][dt]* |
| | L138SiRNA322 as | |
| 4 | siRNA2 | [symbol] [symbol] [dt][dt]* |
| | L138SiRNA131 | |
| 5 | siRNA2 | acuagaaugagauaacaug[dt][dt]* |
| | L138SiRNA131_as | |
| 6 | siRNA3 | cugacaucuccuuucaaac[dt][dt]* |
| | L138SiRNA75 | |
| 7 | siRNA3 | guuugaaaggagaugucag[dt][dt]* |
| | L138SiRNA75_as | |

*dinucleotide 3′-overhangs (can facilitate RISC loading)
**sense sequences are highlighted within the MB3 lincRNA sequence

### Example 2—siRNA Silencing of MB3 lincRNA

The effect of MB3 lincRNA on medulloblastoma tumorigenicity was tested via siRNA silencing of this RNA. To this end, siRNA molecules for silencing MB3 lincRNA where designed and tested. Three siRNA molecules were found to exhibit significant inhibitory effects compared to control: siRNA1 (SEQ ID NOs: 2-3); siRNA2 (SEQ ID NOs: 4-5) and siRNA3 (SEQ ID NOs: 6-7).

Experimental Procedure:

DAOY and UW228-2 medulloblastoma cell lines were transfected with a total of 100 nM siRNA (33.3 nM from each siRNA of the siRNAs set forth in SEQ ID NOs: 2-7) and then incubated for 48 h. The siRNA transfection mixture contained medium without serum, Hiperfect transfection reagent and the siRNAs. Next, RNA was extracted from the cells and reverse transcribed to cDNA. Relative expression levels of MB3 lincRNA in cells transfected with the siRNAs set forth in SEQ ID NOs: 2-7 compared to cells transfected with control scrambled siRNAs (“si negative”, non-targeting pool siRNA) and cells treated with medium only (“MEM control”) were examined by qRT-PCR. The expression level of the MEM control group was set as “1”, and expression levels of the si-negative group and the group transfected with the test siRNAs were calculated accordingly.

TABLE 1B
Control sequences*

| SEQ. | | |
|---|---|---|
| ID. | | |
| No. | Name | Sequence |
| 15 | Si scramble 1 | ACCAAAUGUACAGCUGAUU |
| 16 | Si scramble 2 | ACCAAAUGUACAACACACU |
| 17 | Si scramble 3 | ACCAAAUGUACAAAAGACU |
| 18 | Si scramble 4 | ACCAAAUGUACAAAAGGAU |

*a mix of the four scrambled RNA sequences, which do not bind human sequences, was used as a control.

Results:

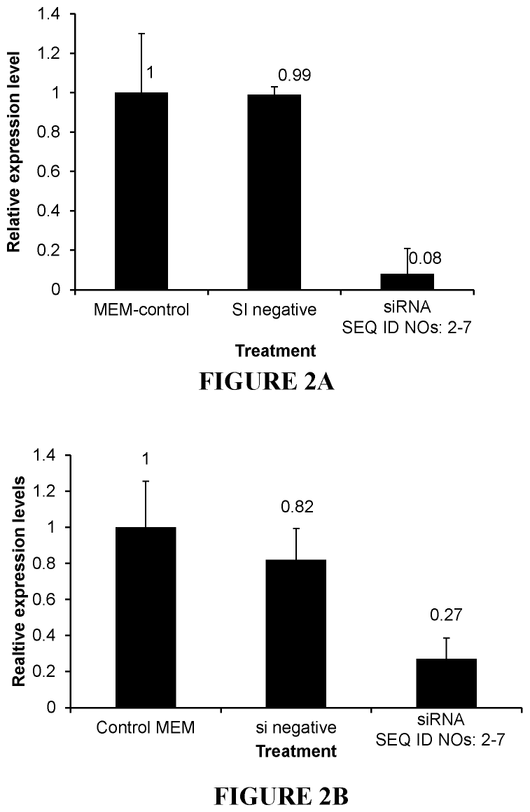

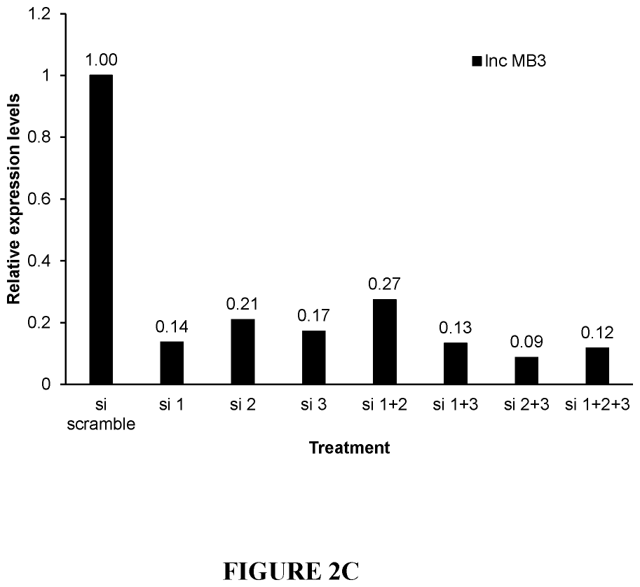

DAOY cells transfected with a composition comprising all three siRNAs set forth in SEQ ID NOs: 2-7 (siRNA1 (SEQ ID NOs:2-3); siRNA2 (SEQ ID NOs:4-5); and siRNA3 (SEQ ID NOs:6-7)) exhibited a significant inhibition of MB3 lincRNA expression, compared to DAOY cells transfected with the control scrambled siRNAs (FIG. 2A; Student's T test pv=5×10⁻⁵ compared to control-negative si. n=3).

UW228-2 cells transfected with a composition comprising all three siRNAs set forth in SEQ ID NOs: 2-7 also exhibited a significant inhibition of MB3 lincRNA expression, compared to UW228-2 cells transfected with the control scrambled siRNAs (FIG. 2B; T test pv=0.005 compared to control-MEM n=3. DAOY cell line *p=0.0018 compared to control-si scramble. In UW-228-2 cell line **p=0.008 compared to control-si scramble).

In a further experiment, the silencing effects of each siRNA alone (siRNA1, siRNA2 and siRNA3, as detailed above) and of different combinations of these siRNAs were tested by RT-PCR:

Experimental Procedure:

DAOY cells were transfected with 33 nM of one of the siRNAs or with their combinations using HiPerFect® transfection reagent and incubated for 48 h. Next, RNA was extracted from the cells and reverse transcribed to cDNA (2 ug). Relative expression levels of MB3 lincRNA in cells treated with the test siRNAs compared to cells treated with control scrambled siRNAs (non-targeting pool siRNA, as detailed above) were examined by qRT-PCR.

Results:

The results are summarized in FIG. 2C. All three siRNAs that were tested, as well as their combinations, significantly inhibited the expression of MB3 lincRNA compared to the control scrambled sequence. SiRNA1 alone had the highest silencing effect. Under the conditions that were tested the combinations did not significantly increase the silencing effect compared to each siRNA alone.

### Example 3—siRNA Inhibits Cellular Invasion

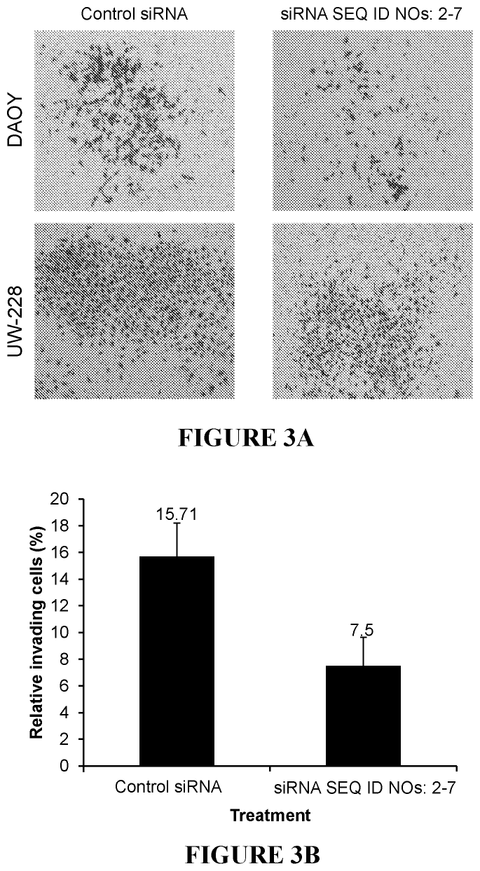

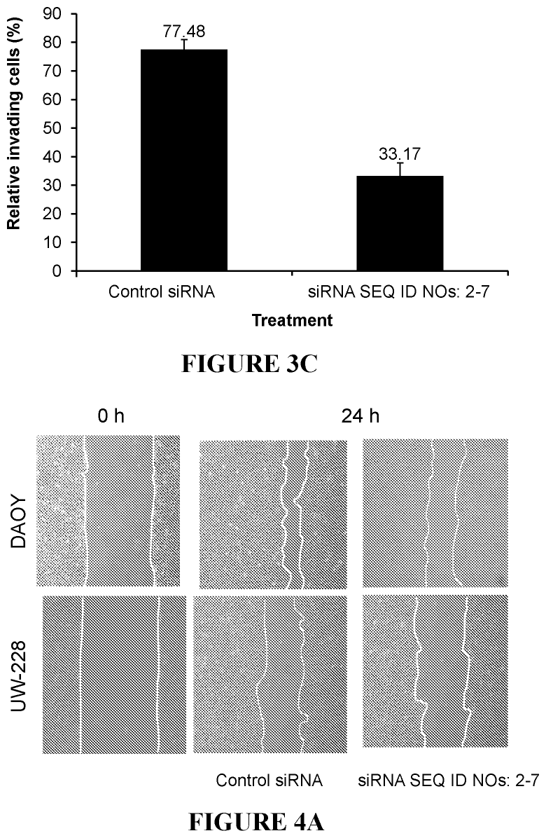

Cell invasion was measured by the following invasion assay: DAOY and UW-228 medulloblastoma cells were incubated (for 48 hours) with an siRNA composition comprising the siRNAs set forth in SEQ ID NOs: 2-7 or a control siRNA composition, then 100,000 treated cells were plated on Matrigel (1 mg/ml) coated transwells, where the bottom of each transwell is an 8 μm pore membrane. 24 hours post plating, cells that crossed (invaded) the membrane were fixed with 4% PFA, stained with 1% crystal violet and counted under a microscope (FIGS. 3A-C). Cells were counted using ImageJ software.

The experimental protocol was carried out in three (3) replicates. Five random fields, ×10 magnification per well, of the invading cells were counted under light microscopy. Data are presented as relative invading cells: the number of invading cells per transwell insert compared to the number of expected invading cells per transwell insert. The latter corresponds to the number of cells initially seeded (1×10⁵ cells per well; 100%).

More particularly, invasion was calculated as follows: the average number of cells in several random fields within an insert was determined, then divided by the area of the microscope viewing field (cm²) and then multiplied by the entire area of the transwell insert (cm²), resulting in the total number of cells per insert, which in reference to the number of cells initially seeded, provided the percent (%) invasion. Area of microscope viewing field=0.0297 cm²; Transwell insert area=0.47 cm².

As shown in FIG. 3B, the percentage of invading DAOY cells after treatment with the siRNAs set forth in SEQ ID NOs: 2-7 was less than half than the percentage of invading DAOY cells treated with the control siRNAs (7.5% compared to 15.7%). The reduction of invasion induced by the siRNA composition according to the present invention was significant (p=0.042).

As shown in FIG. 3C, the percentage of invading UW-228 cells after treatment with the siRNAs set forth in SEQ ID NOs: 2-7 was less than half than UW-228 cells treated with the control siRNA (33.17% compared to 77.48%). The reduction of invasion induced by the siRNA composition according to the present invention was significant (p=3.3×10⁻⁶). The results indicate that inhibiting MB3 lincRNA is associated with inhibition and/or suppression of cellular invasion.

### Example 4—siRNA Inhibits Cellular Migration

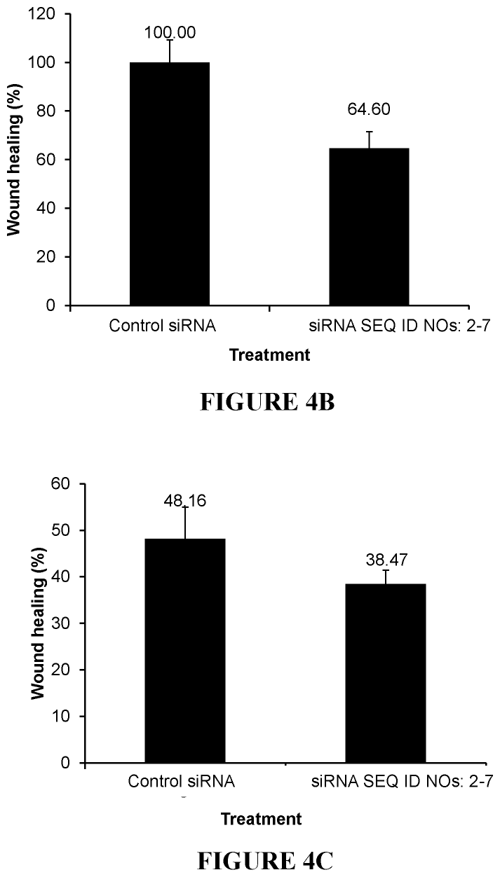

Cell migration was examined by wound healing assays. A control siRNA composition or an siRNA composition comprising the siRNAs set forth in SEQ ID NOs: 2-7 (treatment) were added to medulloblastoma cell lines, DAOY and UW-228, grown to confluent monolayer cell cultures. Cells were starved overnight by using a serum free medium. A scratch in the monolayer was made using a micropipette tip. Cells were then washed by PBS and a complete growth medium was added. After 24 h the distance across the wound/scratch was measured relative to the distance at time 0 (FIGS. 4A-C). In brief, 5 random fields of each cell culture (in a well) were imaged before (0 h) and at 24 h (24 h) after creating the wound by a light microscope (exemplary images are shown in FIG. 4A). In each image, the width of the wound was measured in five random locations. The percentage of migration was calculated using the formula:

$$\% migration = \frac{average wound width 0 h - average wound width 24 h}{average wound width 0 h} \times 100$$

It is noted that the percentage of area/distance/coverage was measured during a time window shorter than the cells' multiplication time (which is 36 hours).

As shown in FIG. 4B, the percentage of wound healing in DAOY cells after treatment was 64.6% relative to control (100%). The reduction in wound healing induced by the siRNA composition was significant (n=3; p=1.8×10⁻¹⁹).

As shown in FIG. 4C, the percentage of wound healing in UW-228 cells after treatment was about 10% less than control (38.47% compared to 48.16%). The reduction of migration induced by the siRNA composition according to the present invention was significant (p=0.005). The results indicate that inhibiting MB3 lincRNA is associated with inhibition and/or suppression of the incidence of cellular migration.

### Example 5—MB3 lincRNA Over-Expression Enhances Cellular Invasion

Enhanced expression of MB3 lincRNA was obtained in medulloblastoma cell lines, DAOY and UW-228, using a plasmid comprising the MB3 lincRNA set forth in SEQ ID NO: 1. An empty pcDNA3 plasmid (a plasmid with no insert) was used as control.

Cell invasion was measured by applying the following invasion assay: DAOY and UW-228 cells over-expressing MB3 lincRNA or control cells containing the empty plasmid were plated on matrigel (1 mg/ml) coated transwells, where the bottom of each transwell is an 8 μm pore membrane. 24 hours post plating, cells that crossed (invaded) the membrane were fixed with 4% PFA (paraformaldehyde), stained with 1% crystal violet and counted under a microscope. Cells were counted using ImageJ software.

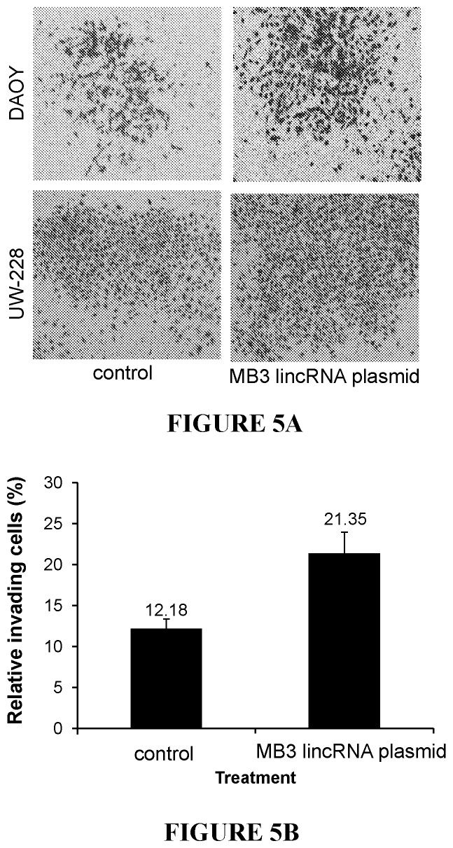

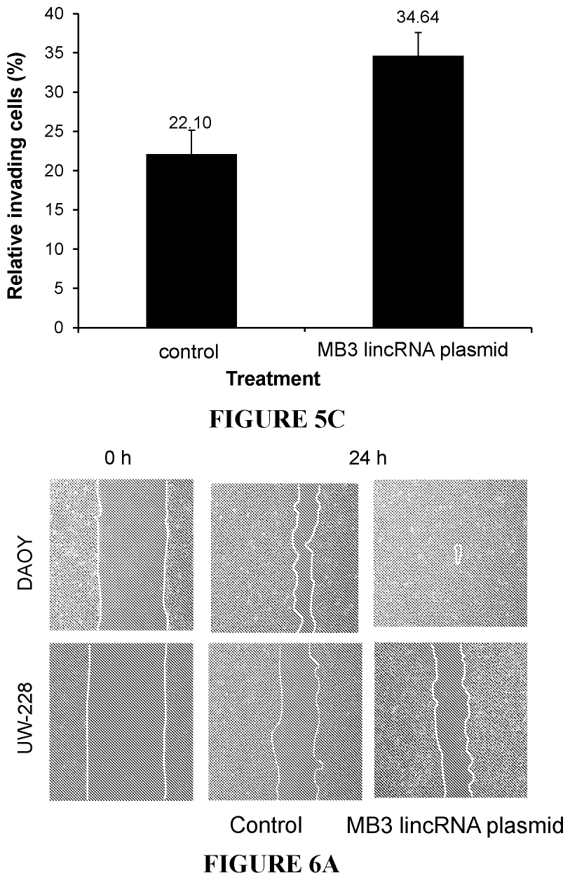

FIG. 5A shows images of invading (stained) cells in selected transwells. FIG. 5B and FIG. 5C show a graphical quantitative representation of the results (DAOY and UW-228 cells, respectively). Data are presented as relative invading cells, calculated as detailed in Example 3 above.

As shown in FIG. 5B, the percentage of invading DAOY cells over-expressing MB3 lincRNA was 21.35% where the percentage of invading control DAOY cells (containing an empty plasmid) was much lower (12.18%). Data is reported as means±SD of 3 repeats. p=0.023.

As shown in FIG. 5C, the percentage of invading UW-228 cells over-expressing MB3 lincRNA was more than 10% higher than control (34.64% compared to 22.10%). p=0.03.

### Example 6—MB3 lincRNA Over-Expression Enhances Cellular Migration

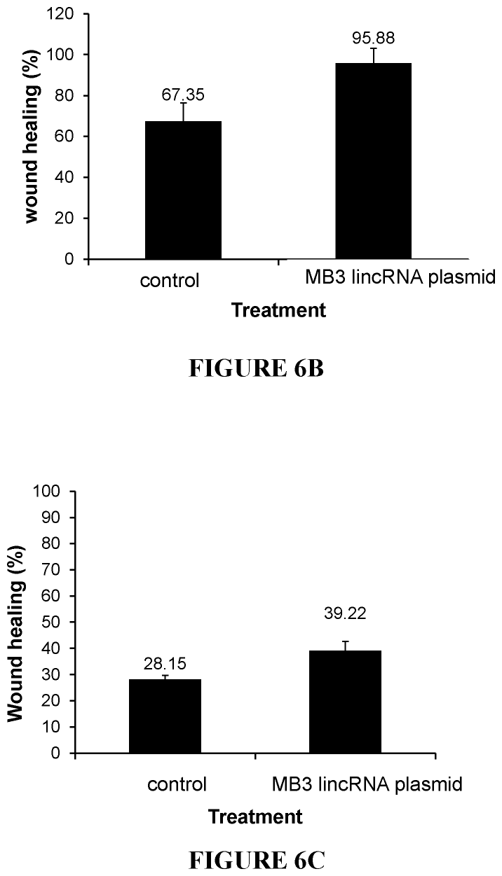

Cell migration was examined in cells over-expressing MB3 lincRNA relative to control cells (obtained as described in Example 5 above) by wound healing assays as described in Example 4. Cells over-expressing MB3 lincRNA or control cells were grown to a confluent monolayer, a scratch was made, and 24 h thereafter the distance across the wound/scratch was measured (FIG. 6A).

As shown in FIG. 6B, the percentage of wound healing obtained with DAOY cells over-expressing MB3 lincRNA was higher compared to control (95.88% compared to 67.35%). The difference in wound healing was significant (n=3; p=6.2×10⁻²²).

As shown in FIG. 6C, the percentage of wound healing obtained with UW-228 cells over-expressing MB3 lincRNA was more than 10% higher compared to control (39.22% compared to 28.15%). The difference in wound healing was significant (n=3; p=1.75×10⁻⁶).

### Example 7—MB3 lincRNA Regulates THSD7A

The mechRNA program was used to predict 63 MB3 lincRNA targets. Of those targets, 6 genes are known to be involved with migration and invasion. Only 3 genes out of the 6 have protein coding transcripts (THSD7A, TSPAN6 and PLXND1). THSD7A was the only gene that was highly induced in patient samples with elevated expression of MB3 lincRNA. Thrombospondin type 1 domain containing 7A (THSD7A) encodes a protein that has been shown to be involved in cell migration, tumor invasion, endothelial blood tube formation (angiogenesis) and in cancer metastasis.

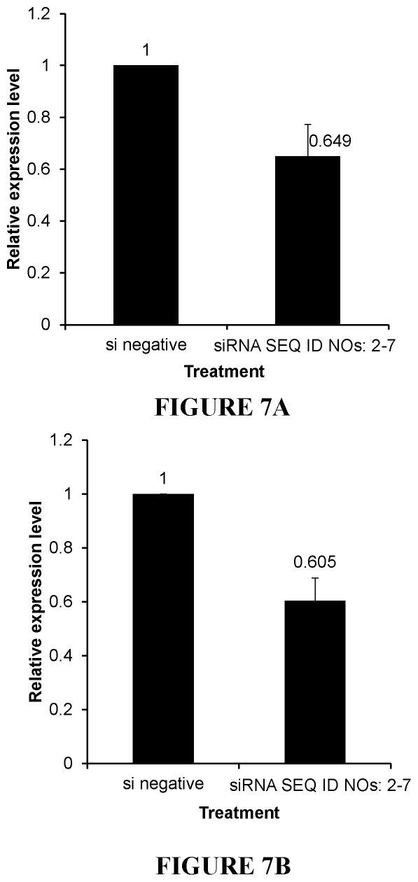

As shown in FIGS. 7A and 7B, THSD7A expression, analyzed by real-time PCR, decreased following silencing of MB3 lincRNA in DAOY cells (p=0.03) and UW-228 cells (p=0.05), respectively. Data are reported as means±SEM of triplicates and statistical significance was determined by Student's T-test.

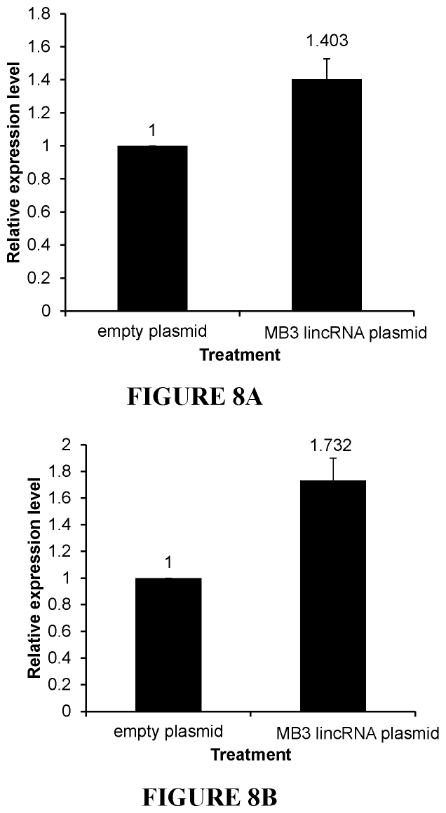

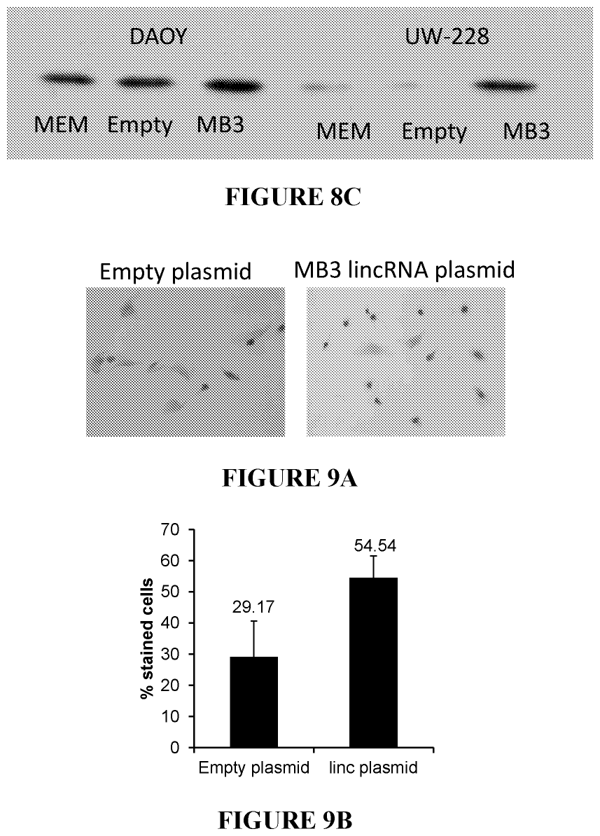

As shown in FIGS. 8A and 8B, THSD7A expression increased following MB3 over-expression in DAOY cells (p=0.033) and UW-228 cells (p=0.06), respectively. Data are reported as means±SEM of triplicates and statistical significance was determined by Student's T-test. Furthermore, the level of THSD7A protein in the medium of DAOY and UW-228 cells over-expressing MB3 lincRNA was increased compared to the medium alone (MEM) and to the medium of cells transfected with an empty plasmid (FIG. 8C).

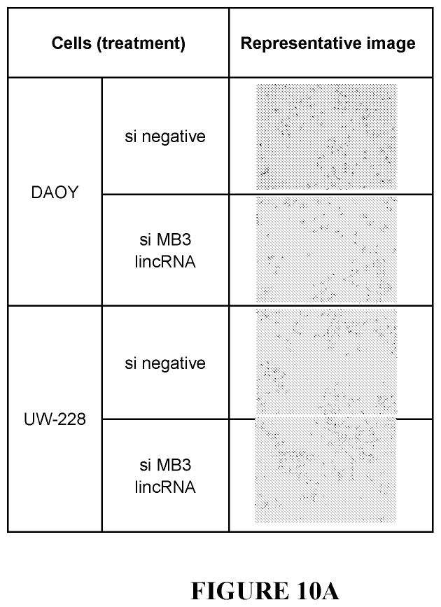

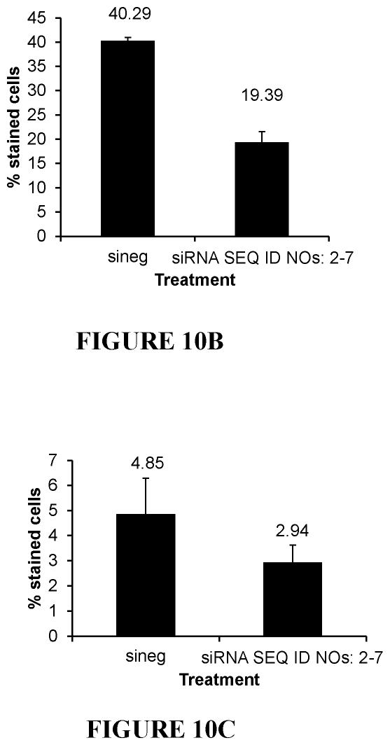

Immunohistochemistry (IHC) with anti-THSD7A (Human Protein Atlas antibodies) and goat anti-rabbit (Jackson Immuno Research Laboratories) revealed a significant elevation of THSD7A in cells over-expressing MB3 lincRNA (FIGS. 9A-B). The relative percentage of stained cells is reported as means±SEM of triplicates, statistical significance was determined by Student's T-test (p=0.041) compared to empty plasmid. As shown in FIGS. 10A-C, THSD7A protein expression decreases following silencing of MB3 lincRNA in DAOY (p=0.02) (FIG. 10B) and UW228 (p=0.22) (FIG. 10C) cells. The stained cells were counted using ImageJ software. Data in FIGS. 10B-C is reported as means±SEM of triplicates where statistical significance was determined by Student's T-test. It can be seen that in control UW-228-2 cells, the percentage of stained cell was very low. Therefore, silencing had little effect on THSD7A protein expression in this type of cells.

Soluble THSD7A is the functional form that promotes endothelial cell migration and significantly increases the phosphorylation level of Focal Adhesion Kinase (FAK).

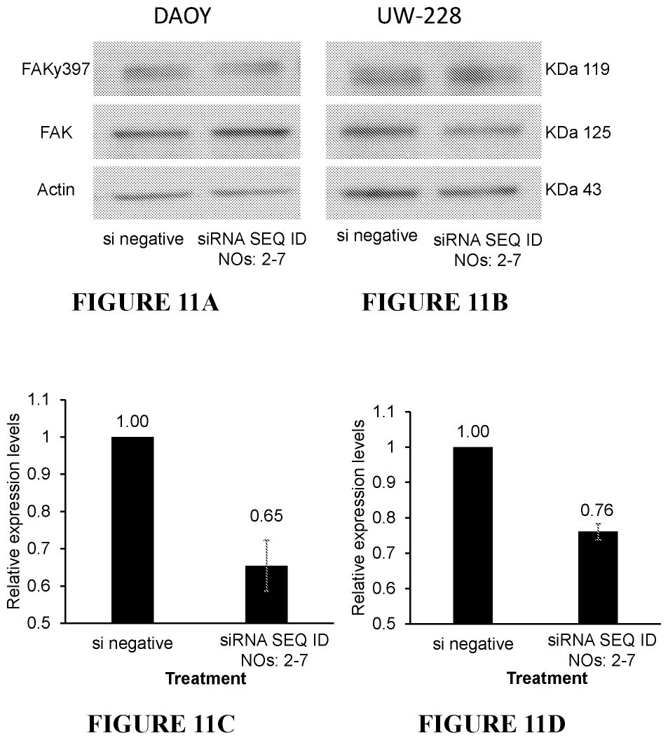

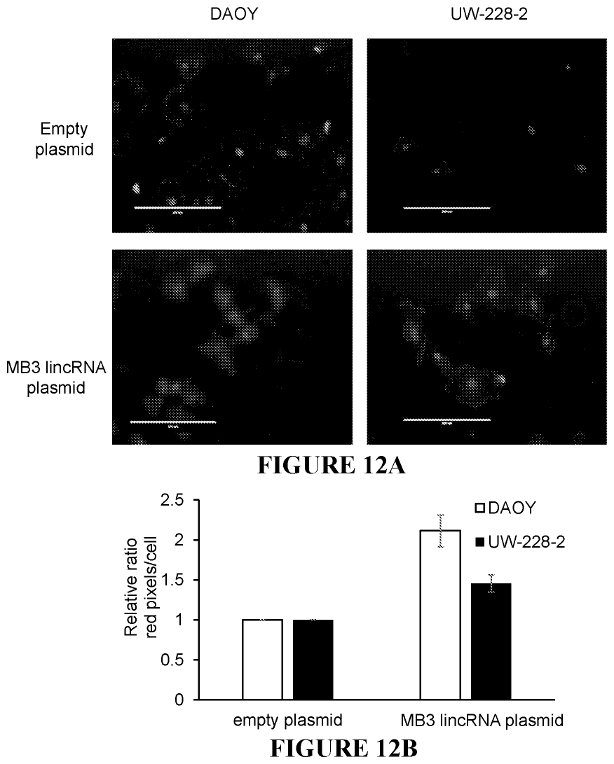

Western blot analysis of FAK and phosphorylated FAK (phospho-FAK^{Y397}) following transfection with the siRNAs set forth in SEQ ID NOs: 2-7 or with a control siRNA composition was carried out. Treatment with the siRNAs set forth in SEQ ID NOs: 2-7 resulted in decreased levels of phosphorylated FAK (phospho-FAKy397, FIGS. 11A-11B) compared to the levels of phosphorylated FAK following treatment with the control siRNA composition. Phospho-FAK^{Y397} molecular weight is 119 kDa, whereas FAK molecular weight is 125 kDa. Actin served as a house keeping gene and is indicated by 43 kDa.

FIGS. 11C-11D show a graphical representation of the relative expression of phospho-FAK^{Y397} relative to FAK (ratio of phospho-FAK^{Y397}/FAK) in cells treated with the siRNAs set forth in SEQ ID NOs: 2-7 or with the control siRNA composition. The ratio of phospho-FAK^{Y397}/FAK in cells treated with the control siRNA composition was set as “1”, and the relative expression in the cells treated with the siRNAs set forth in SEQ ID NOs: 2-7 was calculated relative to the control. Data are reported as means±SEM of triplicates and statistical significance was determined by Student's T-test. In DAOY cells p=0.026 compared to control—si scramble. In UW-228-2 cells p=0.009 compared to control—si scramble.

The effect of over-expression of MB3 lincRNA on phosphorylation of FAK was tested by immunofluorescence (IFC) using anti-FAK (phospho Y397; abcam) and cy3 goat anti-rabbit (red) (Jackson Immuno Research Laboratories). Over-expression of MB3 lincRNA resulted in an increase in FAK phosphorylation at Tyr397 (FIGS. 12A-12B). Nucleus was stained with DAPI to represent each cell in the image and p-FAK was stained with CY3 (red). The relative ratio of red pixels/cell between treatments was calculated. The red pixels were counted using ImageJ software. Data are reported as means±SEM of triplicates and statistical significance was determined by Student's T-test. In DAOY cells p=0.015 compared to control—empty plasmid. In UW-228-2 cells p=0.035 compared to control—empty plasmid.

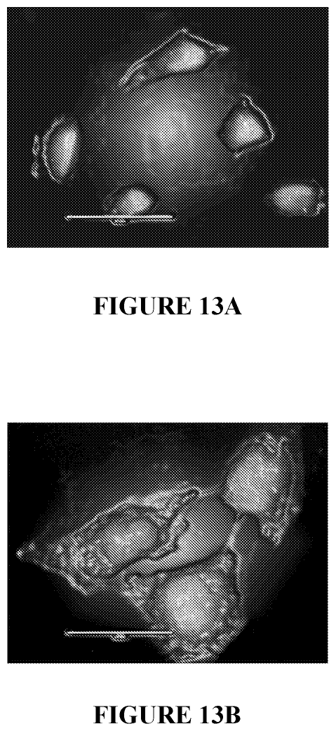

MB3 lincRNA in the cytosol binds THSD7A untranslated region and regulates it. Elevated THSD7A increases FAK phosphorylation which in the nucleus advances proliferation and in the cytosol connects integrins at the leading edge of the cell to form lamellipodia. After recruitment of more proteins filopodia are formed. The cells use this structure as an anchor for moving over the extracellular matrix. Immunofluorescence (IFC) of THSD7A and phospho-FAK(ser 722) following over-expression of MB3 lincRNA revealed that the cells' defined shape changes upon MB3 lincRNA over-expression to developing podia (FIGS. 13A and 13B, respectively).

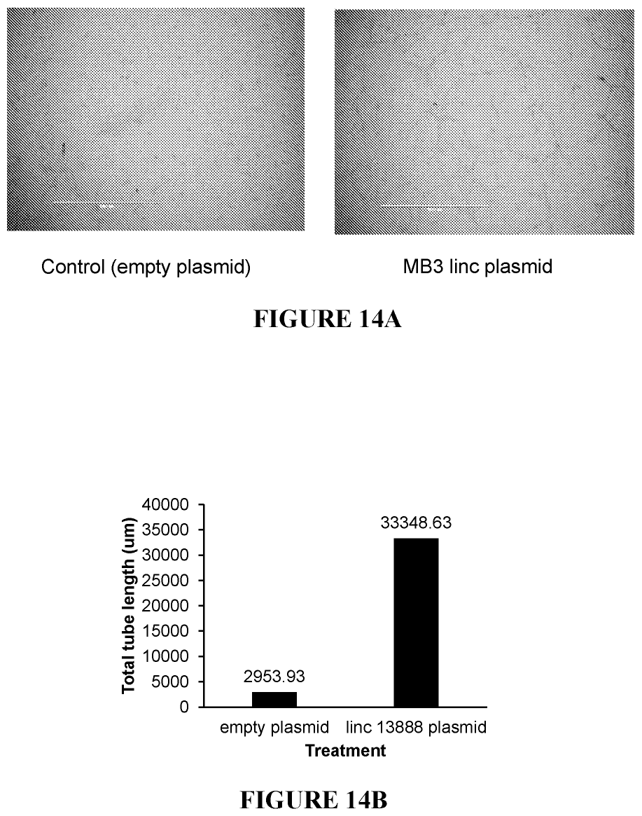

Soluble THSD7A is released from cells to promote angiogenesis and tube formation. The formation of new blood vessels is essential for the growth, development and regeneration of tumors. Tube formation assay monitors the development of blood vessels, by measuring the angiogenesis stage of endothelial cell reorganization to form tube or capillary-like structures, using an extracellular matrix such as Matrigel as support. Immortalized endothelial cells were mixed with a conditioned medium of UW-228 cells over-expressing MB3 lincRNA or containing an empty plasmid, and plated on a basement membrane matrix. The endothelial cells formed capillary-like structures in response to angiogenic signals found in the conditioned medium, as shown in FIG. 14A (right panel) and further summarized in FIG. 14B. Tube structure corresponds to endothelial cells construct lumen-containing tubules visualized using a phase contrast inverted microscope. FIGS. 14A and 14B demonstrate the increase in angiogenesis ability following over-expression of MB3 lincRNA (p=0.05).

### Example 8—Expression of MB4 lincRNA in Medulloblastoma Tumor Samples

Dataset EGAD00001003279 containing data from 64 medulloblastoma patient samples was downloaded from the European Genome—phenome Archive (EGA), together with information on the four core medulloblastoma molecular groups. The RNA sequences were aligned to the human genome (ucsc hg19) using Bowtie 2 program. Non-unique alignments were removed and the number of RNA fragments falling on lincRNAs were counted.

RNAseq results normalization: results were normalized using reads per kilobase transcript per million reads (RPKM), where RPKM is a length normalization and is calculated as follows:

RPKM=[no. of counts/(total count×transcript length|)]×10⁹.

The normalization analysis produced an unbiased estimate of the mean of the genes' expression.

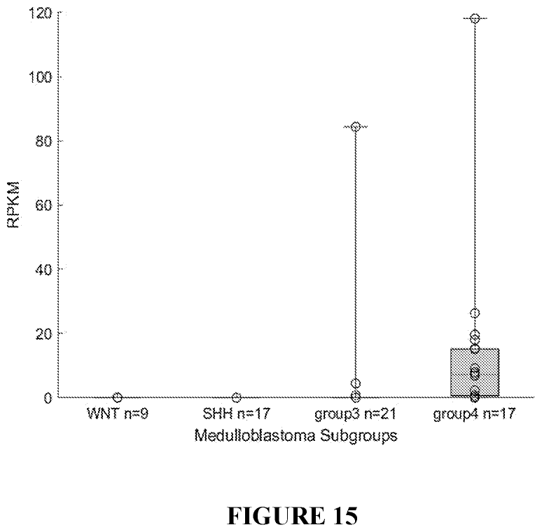

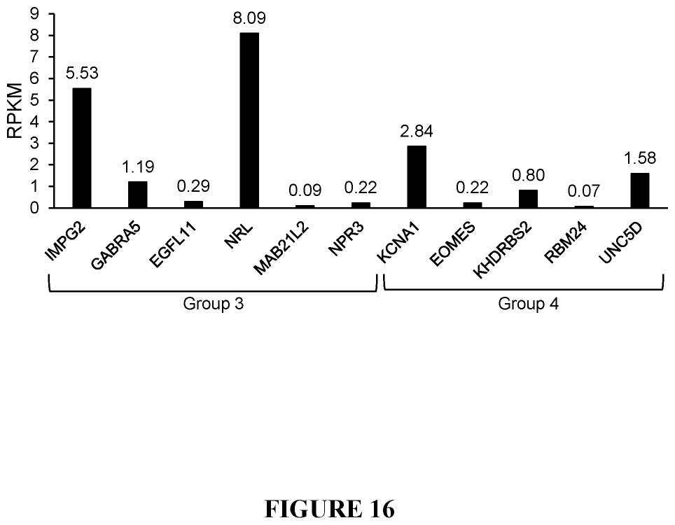

The analysis showed that the expression of the lincRNA set forth as SEQ ID NO: 8 (Table 2), also identified as “MB4 lincRNA”, is significantly higher in medulloblastoma group 4 compared to the other core groups (FIG. 15) (p<0.05 by Kruskal-Wallis in comparison to WNT, SHH and group 3), with the exception of one outlier sample in Group 3. This sample was further analyzed using NanoString nCounter, which is a molecular classification method that uses an expression pattern of 5-6 medulloblastoma subgroup-specific signature genes to determine tumor core group. Detailed NanoString assay gene expression analysis of the outlier sample showed that this sample has features of group 3 (high expression of IMPG2 and NRL) and group 4 (high expression of KCNA1 and UCN50) (FIG. 16). Expression of MB4 lincRNA was not detected in the WNT and SHH groups.

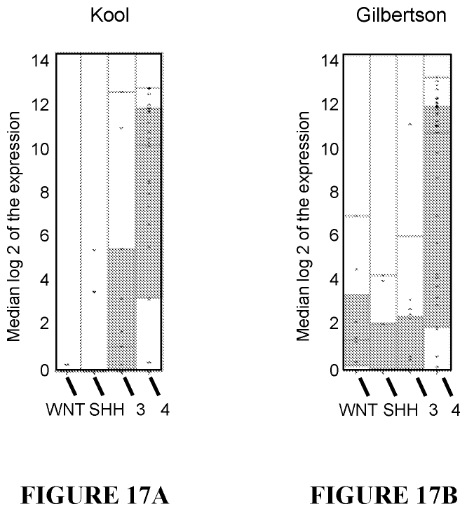

The results were validated in two more datasets of medulloblastoma tumor samples: Kool dataset, n=62, and Gilbertson dataset, n=76 (FIGS. 17A-17B).

Analysis of these datasets showed that the expression of MB4 lincRNA (SEQ ID NO: 8) is high mainly in group 4 compared to the other medulloblastoma core groups. FIGS. 17A and 17B represent the median log 2 of the expression of all samples. The shaded areas in this figure are box plots. The bottom and top hinges of each box are the first and third quartiles (25%-75%), and the band inside each box is the median. Each dot represents results from a single patient. The upper/lower lines outside the boxes are the largest/lowest value no further than 1.5*IQR from the hinge (where IQR is the inter-quartile range, or distance between the first and third quartiles). Data beyond the end of these lines are “outlying” points and are plotted individually. The analysis indicates that median log 2 of the expression is less than 2 in all groups except medulloblastoma group 4, which is above 8, where expression levels above 4 were observed mainly in medulloblastoma group 4.

Nine (9) tumor samples from Kool and Gilbertson datasets that were grouped to group 3 showed high levels of MB4 lincRNA (SEQ ID NO: 8). Expression of MB4 lincRNA was low or non-detectable in the WNT and SHH groups.

The results therefore suggest MB4 lincRNA as a marker of non-WNT/non-SHH medulloblastoma.

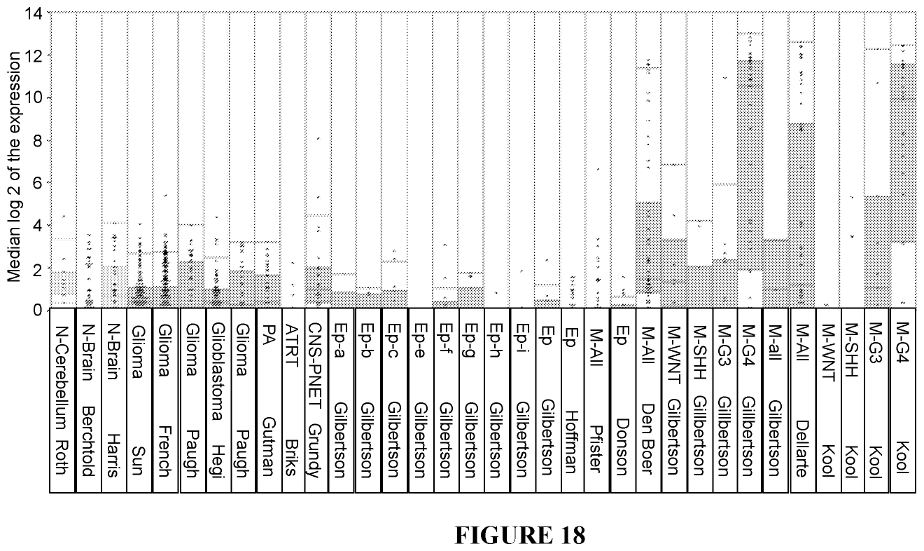

The expression of MB4 lincRNA was further evaluated in publicly available data from microarray studies (FIG. 18) of normal brain (“N-Brain” in FIG. 18), normal cerebellum (“N-Cerebellum”), medulloblastoma (including the Kool and Gilbertson studies noted above) (“M-All”, “M-G3”, “M-G4”, “M-WNT”, “M-SHH”) and brain tumors other than medulloblastoma: glioma, glioblastoma, polycystic astrocytoma (“PA”), ATRT, CNS-PNET and ependymoma (“Ep”). A total of 1389 samples were analyzed. As noted above with respect to FIGS. 17A and 17B, the shaded areas represent box plots. The analysis confirmed that over-expression of MB4 lincRNA is specific to non-WNT/non-SHH medulloblastoma (core groups 3 and 4).

TABLE 2
Sequences

| SEQ. | | |
|---|---|---|
| ID. | | |
| No. | Name | Sequence |
| 8 | MB4 lincRNA | cttccacatcgtggaagctt |
| | (natural) | tgttctttcgctctttgcaa |
| | HGNC Gene | taaatcttgctactgctcac |
| | Symbol: | tctttgggtccacgctgctt |
| | LINC01419 | ttatgagctgtaacactcac |
| | HGNC Full | cgcgaagatctgcagcttca |
| | Gene Name: | ctcccgagccagcgagacca |
| | long | cgaacccaccagaaggaaga |
| | intergenic | aactccgaacacatctgaac |
| | non-protein | atcagaagggcagactccag |
| | coding RNA | acacgccaccttaagagctg |
| | 1419 | taacactcaccgcgagggtc |
| | Location | cacggcttcattcttgaagt |
| | (hg19): | cagtgagaccaagaacccac |
| | chr8: | caattccggacacaatttct |
| | 84315992- | tggctctcagtggcttccat |
| | 84321157 | ggaagttctgcaatcaacca |
| | Strand: + | gcaggagaaccgcttaaacc |
| | | caggaggcggaggttgcagt |
| | | gagccaagtatgcatcactg |
| | | cactccagcctggaagacag |
| | | agtgagaccctgtctcaaca |
| | | aaataaattaaaataaaaaa |
| | | taatatatttttctaactat |
| | | catcccttttccaaatcagg |
| | | aattccccttaagttttcct |
| | | caatttccatggcaatatct |
| | | ttgcatagattcattaagaa |
| | | tttgtcattttaaataaaaa |
| | | atataaagggaactattcat |
| | | taggcaacaaatgccttgtc |
| | | tgaaatatcacatttgagaa |
| | | tgctgctcatttaatcagaa |
| | | aggtacgctactttaaagaa |
| | | ctgaggtccactttctggag |
| | | ccaaaaactcataaatccct |
| | | ctcagaaaaacctgatttgc |
| | | tttgtagggtctcaggttta |
| | | gagatgctgaaaaagatatt |
| | | ttcgttgcagacaaaggacc |
| | | tcagagtatttggagaactt |
| | | tgagaagagaggaattctcc |
| | | caaatgtataggtgtcacag |
| | | gtaaaatacagtcgagagat |
| | | tttcttggactttaattcct |
| | | taaatcaggatagcaaataa |
| | | taggggcttttacaaattca |
| | | atctgtttccttacaaaaat |
| | | tttcagcaaagtaatttcag |
| | | caaattaattttcagcaaag |
| | | taaatgtaagaagacttatg |
| | | tgaaaaattaacattctcca |
| | | tgtatctatgaagccaaacc |
| | | taataaaaccagctttaatt |
| | | tgtgctcaagaatattattt |
| | | cactgagttttcttaaatca |
| | | caaaggggagactgttatga |
| | | aaactgatataaaataaaaa |
| | | aaacaaggaagaaaagtcta |
| | | ctagatgtctctaatggaag |
| | | actgcatttttagaacatat |
| | | ccttataggcgattctagcc |
| | | tttctctgctatttggctct |
| | | cacactctttaccgtgcaga |
| | | taattcacagcaatgcaaaa |
| | | gaatcctcatctatagccat |
| | | gaaaataagttatttgttat |
| | | ttctggtaaaggttcaattg |
| | | acctccccttccaggatgaa |
| | | gaaagtttcatgtctttctg |
| | | catcatttcaactattcctt |
| | | actacatataaatctgcact |
| | | tgttaacttctattttgaat |
| | | tgattgtggcatctgcctgc |
| | | ttccccattaaaactgaata |
| | | aaatctttaacacataaaaa |
| 9 | ASO1 | mG*mU*mG* mU*mC*T* |
| | | G*G*A* G*T*C* T*G*C* |
| | | mC*mC*mU* mU*mC |
| 10 | ASO2 | mG*mA*mA* mG*mC*A* |
| | | G*G*C* A*G*A* T*G*C* |
| | | mC*mA*mC* mA*mA |
| 11 | ASO3 | mA*mG*mG* mU*mC*C* |
| | | T*T*T* G*T*C* T*G*C* |
| | | mA*mA*mC* mG*mA |
| 12 | Corresp tASO1 | Gcagactcca |
| 13 | Corresp tASO2 | Gcatctgcct |
| 14 | Corresp tASO3 | Gcagacaaag |

*Phosphorothioate bonds; m = 2′-O-Me RNA base

### Example 9—Detecting Tumor Recurrence by MB4 lincRNA Liquid Biopsy Preceding MRI Detection of the Tumor

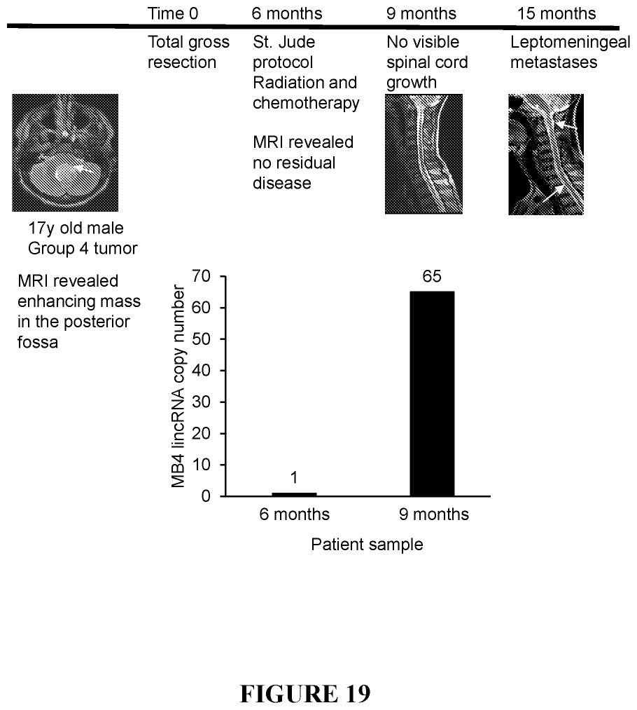

A 17-year old male patient. MRI revealed an enhanced mass in the posterior fossa, classified as group 4. Six months after tumor gross resection and 4 rounds of St. Jude treatment protocol, another MRI revealed no residual disease. No MB4 lincRNA was detected in the patient's blood, as measured by real-time PCR. Nine (9) months after surgery, there was no visible spinal cord growth. However, high levels of MB4 lincRNA were detected in his blood. Leptomeningeal metastases were diagnosed in the MRI 5 months after the detection of MB4 lincRNA in the blood. This experiment highlights the utility of detecting MB4 lincRNA in the blood prior to clinical diagnosis of tumors by MRI (FIG. 19).

### Example 10—MB4 lincRNA in Random Pediatric Cancer Samples

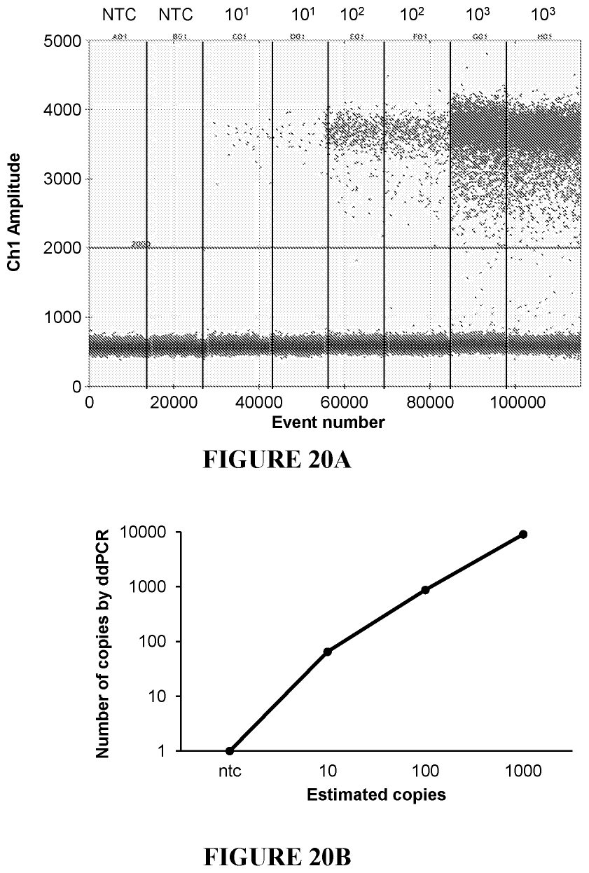

Highly sensitive Droplet Digital PCR (ddPCR) facilitates detection and quantification of low levels of DNA by partitioning DNA samples into water-in-oil droplets. In order to utilize ddPCR for quantifying the levels of MB4 lincRNA in plasma samples, it was first important to determine the sensitivity of detection and quantification, and the linearity of the droplet digital technique. To this end, MB4 lincRNA (SEQ ID NO: 8) template were diluted 10-fold, starting from 10³ copies down to 10 copies. In addition, non-template control (NTC) was evaluated (FIG. 20A). Log scale curve of the copy numbers detected by ddPCR resembled the estimated number of copies and the 10× dilution calculated by nanodrop concentration and amplicon weight (FIG. 20B).

Next, blood samples were received from medulloblastoma pediatric patients, non-medulloblastoma oncology pediatric patients, and non-oncology pediatric patients. Plasma was separated using ficoll and stored at −80° C. RNA was extracted from 0.5 ml plasma using Direct-zol™ MicroPrep RNA extraction. RNA was converted to cDNA using high capacity cDNA transcription. Absolute quantification of MB4 lincRNA transcript was measured by digital PCR and read by Qx100 droplet reader. A cutoff of 10 copies was set as normal according to 5 normal pools with 4-12 plasma samples of non-oncology pediatric patients.

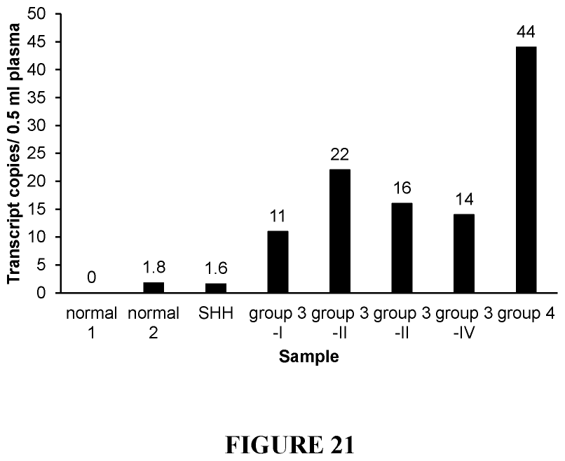

FIG. 21 shows MB4 lincRNA levels below the cutoff in plasma samples of non-oncology pediatric patients (normal) (n=2) and in a plasma sample of a medulloblastoma SHH patient (n=1). The figure further shows MB4 lincRNA levels above the threshold level that was set (above 10 copies) in plasma samples of medulloblastoma group 3 (n=4) and medulloblastoma group 4 (n=1) patients.

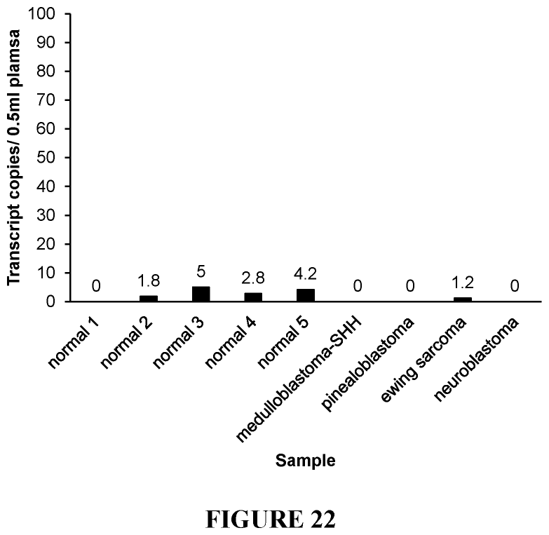

FIG. 22 shows MB4 lincRNA levels below the cutoff in plasma samples of non-oncology pediatric patients (normal) (n=5) and in plasma samples of random pediatric cancer patients diagnosed with medulloblastoma SHH (n=1), pineoblastoma (n=1), Ewing sarcoma (n=1) and neuroblastoma (n=1).

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1-24. (canceled)

25. A synthetic double-stranded RNA targeting MB3 lincRNA or a vector encoding the same, selected from the group consisting of:
(A) a dsRNA comprising a sense strand comprising the sequence set forth in SEQ ID NO: 2 and an antisense strand comprising the sequence set forth in SEQ ID NO: 3;
(B) a dsRNA comprising a sense strand comprising the sequence set forth in SEQ ID NO: 4 and an antisense strand comprising the sequence set forth in SEQ ID NO: 5; and
(C) a dsRNA comprising a sense strand comprising the sequence set forth in SEQ ID NO: 6 and an antisense strand comprising the sequence set forth in SEQ ID NO: 7,
wherein each strand is between 19 to 25 nucleotides in length.

26. A pharmaceutical composition comprising a synthetic dsRNA of claim 25 and a pharmaceutically acceptable carrier.

27. A method for treating medulloblastoma in a subject in need thereof, the method comprising administering to the subject a synthetic dsRNA of claim 25, or a composition comprising the same.

28. A method for treating a subject suffering from medulloblastoma, the method comprising:
(i) classifying the medulloblastoma as Group 3 and/or metastatic medulloblastoma by detecting a level of MB3 lincRNA (SEQ ID NO: 1) above a predefined threshold in a sample from the subject, and administering the subject an MB3 lincRNA inhibitor or a composition comprising the same; or
(ii) classifying the medulloblastoma as non-WNT/non-SHH medulloblastoma by detecting a level of MB4 lincRNA (SEQ ID NO: 8) above a predefined threshold in a sample of the tumor, and administering to the subject an MB4 lincRNA inhibitor or a composition comprising the same.

29. The method of claim 28, wherein the level of the at least one lincRNA is measured by extracting RNA from the sample, reverse-transcribing the RNA to cDNA, and subjecting the cDNA to quantitative amplification for quantifying the at least one lincRNA.

30. The method of claim 28, wherein the sample is a tumor sample.

31. The method of claim 28, wherein the inhibitor is a nucleic acid.

32. The method of claim 31, wherein the nucleic acid is selected from the group consisting of small-inhibitory RNA (siRNA), short hairpin RNA (shRNA), microRNA (miRNA) and antisense oligonucleotide (ASO), or a vector carrying the same.

33. A method of treating medulloblastoma in a subject in need thereof, the method comprising:
(i) measuring a level of MB3 lincRNA in a sample from the subject;
(ii) detecting a level of MB3 lincRNA above a control level; and
(ii) administering to the subject an MB3 lincRNA inhibitor.

34. The method of claim 33, wherein the sample is a tumor sample.

35. The method of claim 33, wherein the MB3 lincRNA inhibitor is a nucleic acid targeting MB3 lincRNA.

36. The method of claim 35, wherein the nucleic acid targeting MB3 lincRNA is any one of a small-inhibitory RNA (siRNA), short hairpin RNA (shRNA), microRNA (miRNA), or antisense oligonucleotide (ASO), or a vector carrying the same.

37. The method of claim 36, wherein the nucleic acid targeting MB3 lincRNA is an siRNA or a vector encoding the same selected from the group consisting of:
(a) siRNA with a sense sequence as set forth in SEQ ID NO: 3 and an antisense sequence as set forth in SEQ ID NO: 3;
(b) siRNA with a sense sequence as set forth in SEQ ID NO: 4 and an antisense sequence as set forth in SEQ ID NO: 5;
(c) siRNA with a sense sequence as set forth in SEQ ID NO: 6 and an antisense sequence as set forth in SEQ ID NO: 7;
and combinations thereof.
